(19) United States
(12) Patent Application Publication
(10) Pub. No.: US 2018/0187169 A1
(43) Pub. Date: Jul. 5, 2018

(54) THIOLASE VARIANTS AND METHODS OF USE THEREOF

(71) Applicant: Massachusetts Institute of Technology, Cambridge, MA (US)

(72) Inventors: Yekaterina Tarasova, Cambridge, MA (US); Brian Bonk, Cambridge, MA (US); Kristala L. Jones Prather, Milton, MA (US); Bruce Tidor, Lexington, MA (US)

(73) Assignee: Massachusetts Institute of Technology, Cambridge, MA (US)

(21) Appl. No.: 15/679,090

(22) Filed: Aug. 16, 2017

Related U.S. Application Data
(60) Provisional application No. 62/375,480, filed on Aug. 16, 2016.

(51) Int. Cl. C12N 9/10; C12P 7/62; C12P 7/42; C12P 7/04

(52) U.S. Cl. CPC C12N 9/1029; C12P 7/04; C12P 7/42; C12P 7/625

(57) ABSTRACT

Described herein are thiolase variants, cells expressing the thiolase variants, and methods of their use for the biosynthesis of desired products.

### RELATED APPLICATIONS

This application claims the benefit under 35 U.S.C. § 119(e) of U.S. provisional application No. 62/375,480, filed Aug. 16, 2016, which is incorporated by reference herein in its entirety.

### GOVERNMENT SUPPORT

This invention was made with Government support under Grant No. R01 GM082209 awarded by the National Institutes of Health. The Government has certain rights in the invention.

### FIELD OF THE INVENTION

This invention relates to thiolase variants having altered activities, such as increased selectivity ratios, recombinant cells expressing the thiolase variants, and methods of using such cells for the biosynthesis of desired products.

### BACKGROUND OF INVENTION

Microbial fermentation affords many advantages for the synthesis of commodity and specialty chemicals over more traditional methods. These include mild reaction conditions, avoidance of harsh and toxic chemicals, and the ability to utilize renewable feedstocks (Keasling, 2009). Advances in metabolic engineering and synthetic biology now allow for fast construction and manipulation of heterologous pathways in canonical production host strains (Lee et al., 2012). Although a wide variety of useful compounds have been synthesized using biological systems, few of these pathways have been commercialized. For a given pathway to be commercially viable, the process must produce the desired product in high yield, at a high titer and with high productivities.

### SUMMARY OF INVENTION

Aspects of the present disclosure provide thiolase variants comprising an amino acid substitution at a position corresponding to M157 and/or M288 of SEQ ID NO: 1 (PhbA from Z. ramigera), wherein the thiolase variant has greater than 40% amino acid identity to SEQ ID NO: 1 or wherein the region within 10 angstroms of the active site of the thiolase variant has greater than 75% amino acid identity to the corresponding region of SEQ ID NO:1.

Aspects of the present disclosure provide thiolase variants comprising an amino acid substitution at one or more positions corresponding to V57, Q87, L88, S91, L93, D146, L148, T149, D150, M157, M288, N316, I350, S353, L377, I379, or Q64 of SEQ ID NO: 1. In some embodiments, the the amino acid substitution is at position L88, M157, M288, and/or L377. In some embodiments, the amino acid substitution is selected from the group consisting of L88S, M157A, M157G, M157S, M288A, M288G, and M288S.

Aspects of the present disclosure provide thiolase variants comprising an amino acid substitution at one or more positions corresponding to V57, R88, L89, S92, L94, A148, L149, H150, D151, M158, M290, N318, A320, F321, I352, T355, M379, I381, I387, or Y66 of SEQ ID NO: 2. In some embodiments, the amino acid substitution is at position Y66, M158, and/or M290. In some embodiments, the amino acid substitution is selected from the group consisting of M158A, M158G, M158S, M290G, and M290S.

Aspects of the present disclosure provide thiolase variants comprising an amino acid substitution at one or more positions corresponding to M156 or M287 of SEQ ID NO: 3. Aspects of the present disclosure provide thiolase variants comprising an amino acid substitution at one or more positions corresponding to M157 or M289 of SEQ ID NO: 4.

In any of the embodiments described herein, the thiolase variant has an enhanced selectivity ratio as compared to a thiolase that does not comprise the amino acid substitution. In some embodiments, the enhanced selectivity ratio corresponds to production of an increased ratio of one or more C6 products relative to one or more C4 products. In some embodiments, the C6 product is 3HH-CoA. In some embodiments, the C4 product is 3HB-CoA.

Other aspects provide a nucleic acid encoding any of the the thiolase variants described herein. Other aspects provide vectors comprising any of the nucleic acids described herein.

Yet other aspects provide cells that recombinantly expresses any of the thiolase variants described herein. In some embodiments, the cell further recombinantly expresses any one or more enzymes selected from the group consisting of: (a) a Coenzyme A activator enzyme; (b) a NADPH dependent reductase; and (c) a thioesterase. In some embodiments, the Coenzyme A activator enzyme is Pct from Megasphaera elsdenii. In some embodiments, the NADPH dependent reductase is PhaB from Cupriavidus necator. In some embodiments, the thioesterase is TesB from Escherichia coli.

In some embodiments, the cell further recombinantly expresses any one or more enzymes selected from the group consisting of: (a) an enoyl-CoA reductase; (b) an enoyl-CoA dehydratase; (c) a PHA polymerase; (d) an alcohol or aldehyde dehydrogenase; (e) a carboxylic acid reductase; and (f) a hydroxylase. In some embodiments, the enoyl-CoA reductase is Ter from Treponema denticola. In some embodiments, the enoyl-CoA dehydratase is PhaJ4b from Cupriavidus necator. In some embodiments, the PHA polymerase is PhaC2 from Rhodococcus aetherivorans. In some embodiments, the carboxylic acid reductase is Car from Nocardia iowensis. In some embodiments, the hydroxylase is AlkBGT from Pseudomonas putida.

In some embodiments, the cell is a bacterial cell, a fungal cell, a plant cell, an insect cell, or an animal cell. In some embodiments, the cell is a bacterial cell. In some embodiments, the bacterial cell is an Escherichia coli cell.

In some embodiments, the cell produces a 3-hydroxalkonic acid (3HA), carboxylic acid, dicarboxylic acid, methyl ketone, hydroxy-carboxylic acid, PHA, keto-acid, aldehyde, alcohol, or alkane. In some embodiments, the cell produces a desired 3HA and a byproduct HA, and wherein the ratio of the desired 3HA to byproduct HA is greater than 1. In some embodiments, the desired 3HA is 3-oxo-hexanoyl-CoA, 3-hydroxy-hexanoic acid, or 3-hydroxy-hexanoate (3HHx). In some embodiments, the byproduct 3HA is acetoacetyl-CoA or 3-hydroxbuytric acid (3HB).

In some embodiments, the desired 3HA is 4-methyl pentanol. In some embodiments, the byproduct is butyrate.

Other aspects provide a cell culture or supernatant collected from culturing one or more of the cells described herein. In some embodiments, the cell culture or supernatant contains at least 0.1 g/L 3HHx. In some embodiments, the 3HHx is further purified from the cell culture or supernatant. In some embodiments, the cell culture or supernatant contains at least 0.1 g/L 4-methyl pentanol. In some embodiments, the 4-methyl pentaol is further purified from the cell culture or supernatant.

Other aspects provide methods comprising culturing any of the cells described herein in cell culture medium. In some embodiments, glucose is added to the cell culture medium. In some embodiments, butyrate is added to the cell culture medium. In some embodiments, isobutyrate is added to the cell culture medium.

Yet other aspects provide methods for producing 3-hydroxy-hexanoic acid or 3-hydroxy-hexanoate comprising culturing any of the cells described herein. Other aspects provide methods for producing 4-methyl pentanol comprising culturing any of the cells described herein.

These and other aspects of the invention, as well as various embodiments thereof, will become more apparent in reference to the drawings and detailed description of the invention. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1A shows a generalized 3-hydroxy acid (3HA) pathway, which is also referred to as CoA-dependent chain elongation or reverse β-oxidation. This pathway consists of four core enzymes: a coenzyme-A (CoA) activating enzyme, which converts a small acid precursor to a CoA thioester; a thiolase, which brings about the condensation of the CoA activated acid and acetyl-CoA; a reductase, which reduces the β-carbonyl of the resulting longer chain intermediate; and a thioesterase, which cleaves the thioester bond of the 3-hydroxyacyl-CoA, releasing free CoASH and the free 3-hydroxyacid. A wide variety of other compounds can be produced by addition of other enzymes that can act on the 3-hydroxyacyl-CoA intermediates, such as enoyl-CoA dehydratases and reductases, and alcohol and aldehyde dehydrogenases. Biosynthesis of longer chain 3HAs and carboxylic acids, as well as ω-carboxylic acids, and longer chain alcohols has been demonstrated (Cheong et al., 2016b; Sheppard et al., 2014). However, a mix of products of variable chain lengths results.

FIG. 1B shows an example of a four-enzyme pathway for the synthesis of poly-3-hydroxybutyrate-co-3-hydroxyhexanoate (poly-3HB-co-3HHx), the composition of which depends on thiolase selectivity. Activation of butyrate by the action of Pct (from M. elsdenii), leads to butyryl-CoA which is then condensed with acetyl-CoA (produced from glucose through glycolysis) by a thiolase, either BktB (from C. necator) or PhbA (from Z. ramigera), to produce 3-oxohexanoyl-CoA. This intermediate is then reduced to 3HH-CoA by an acetoacetyl-CoA reductase PhaB (from C. necator). The thiolase is also capable of condensing two acetyl-CoA molecules, which leads to production of 3HB-CoA upon reduction by PhaB. The 3HA-CoA intermediates are then polymerized into PHAs by PhaC2 (from R. aetherivorans 124).

FIG. 1C shows a schematic diagram of the reaction mechanism of the thiolase. This occurs by a biological Claisen condensation reaction though a sequential bi bi ping-pong mechanism. In addition to other thiolases, this mechanism is similar to that utilized by acetyltransferase and ketosynthase domains of polyketide synthetases. Panel 2 corresponds to Bind 1 and Panel 5 corresponds to Bind 2 on which structure based design calculations described herein were performed.

FIG. 1D shows the atomic nomenclature used throughout the present disclosure.

FIG. 2 shows that four different products can result from the condensation reaction of acetyl-CoA (indicated with “A”) and butyryl-CoA (indicated with “B”) catalyzed by the thiolase. The product formed depends on the order of addition of the acyl-CoAs into the active site of the enzyme. The priming acyl-CoA (A or B) serves as an electrophile at the carbonyl carbon and forms an acyl-enzyme intermediate. The extending acyl-CoA (A or B) in this case acts as a nucleophile after abstraction of an α proton and formation of a carbanion. Self-condensation of two acetyl-CoA molecules results in formation of acetoacetyl-CoA, which are termed the AA condensation product, and subsequent reduction by PhaB leads to the formation of 3-hydroxy-butyrate (3HB). Condensation with butyryl-CoA as the priming acyl-CoA and acetyl-CoA as the extending acyl-CoA forms 3-oxo-hexanoyl-CoA which are termed the BA condensation product, and subsequent reduction by PhaB leads to the formation of 3-hydroxy-hexanoate (3HH) and other products. In this study it was sought to increase the ratio of 3HH to 3HB by increasing the ratio of the BA condensation product relative to the AA condensation product.

FIG. 3A shows a model of the structure of Z. ramigera PhbA thiolase active site during the first binding event (Bind 1, corresponding to step 2 in FIG. 1C). The atoms indicated by arrows show the extra atoms of the butyryl group compared to the acetyl group that must be accommodated in order to preferentially produce 3-oxo-hexanoyl-CoA rather than acetoacetyl-CoA.

FIG. 3B shows a model of the structure of Z. ramigera PhbA thiolase active site during first binding event with residues selected for mutation indicated by arrows.

FIG. 3C shows a model of the structure of Z. ramigera PhbA thiolase active site during second binding event (Bind 2, corresponding to step 5 in FIG. 1C). Atoms indicated with arrows show the extra atoms that must be accommodated in order to preferentially produce 3-oxo-hexanoyl-CoA.

FIG. 3D shows a model of the structure of Z. ramigera PhbA thiolase active site during second binding event (corresponding to step 5 in FIG. 1C) with residues selected for mutation indicated by arrows.

FIG. 4A shows results of the initial screening of Z. ramigera PhbA thiolase variants as selected by the computational methods described herein. Single point mutations in PhbA are shown on the x-axis. The thiolase variants were screened using the previously established 3HA pathway (FIG. 1A), resulting in production of free 3HA in the supernatant. Products were analyzed from cell-free culture supernatants 72 hours post induction using HPLC, and ratios were calculated on a molar basis.

FIG. 4B shows the final concentrations of 3HB (light gray) and 3HH (dark gray) acids produced by cells expressing the indicated thiolase variants 72 hours post induction.

FIG. 4C shows Z. ramigera PhbA thiolase variants screened for PHA biosynthesis (presented in FIG. 1B). Ratios represent the composition of PHA polymers as measured by GC after methanolysis. 3HHx represents the condensation of butyryl-CoA with acetyl-CoA, and 3HB of two acetyl-CoA molecules.

FIG. 4D shows PHA content as a weight percentage of the cell dry weight (CDW) of cells expressing the indicated PhbA thiolase variant. 3HB is shown in light gray, and 3HHx is shown in dark gray.

FIG. 5A shows a model of the structure of the active site of C. necator BktB thiolase during the first binding event (corresponding to step 2 in FIG. 1C). The atoms indicated with arrows show the extra atoms of the butyryl group that must be accommodated compared to the acetyl group in order to preferentially produce 3-oxo-hexanoyl-CoA rather than acetoacetyl-CoA.

FIG. 5B shows a model of the structure of the active site of C. necator BktB thiolase during first binding event with residues selected for mutation indicated with arrows.

FIG. 5C shows a model of the structure of the active site of C. necator BktB thiolase during the second binding event (corresponding to step 5 in FIG. 1C). Atoms indicated with arrows show the extra atoms that must be accommodated in order to preferentially produce 3-oxo-hexanoyl-CoA.

FIG. 5D shows a model of the structure of the active site of C. necator BktB thiolase during second binding event (corresponding to step 5 in FIG. 1C) with residues selected for mutation are indicated with arrows.

FIG. 6A shows the indicated C. necator BktB thiolase variants profiled within the context of PHA biosynthesis. Ratios represent the composition of PHA polymers as measured by GC after methanolysis.

FIG. 6B shows PHA content as a weight percentage of the cell dry weight (CDW) of cells expressing the indicated BktB thiolase variant. 3HB is shown in light gray, and 3HHx is shown in dark gray.

FIG. 7 shows the synthesis of C6 products (in addition to C4 products) solely from glucose by cells overexpressing a trans-enoyl-CoA reductase (ter_{Td}) and reductase (PhaJ4b_{Cn}), in addition to a thiolase, acetoacetyl-CoA reductase, and a PHA polymerase. The left panel shows the ratio of 3HH to 3HB, as measured by gas chromatography after methanolysis. The right panel shows PHA content as a weight percentage of the cell dry weight (CDW). 3HB is shown in light gray, and 3HH is shown in dark gray. The BktB M158A variant resulted in increased selectivity for longer chain products as compared to wild-type BktB.

FIG. 8 presents a protein gel of crude cell lysates from strains expressing the PHA biosynthesis pathway with either wild type BktB or the indicated BktB variant enzymes. Briefly, 1 ml of each culture (48 hours post-induction) was collected, and the supernatant was removed after centrifugation. Cells were resuspended in 0.4 mL His buffer and lysed by bead-beating. Protein concentration was determined by a Bradford assay, and 5 μg total protein/BSA equivalent was loaded onto protein gel. The estimated molecular masses are as follows: BktB=40.9 kDa (shown with an arrow), Pct=55.6 kDa, PhaB=26 kDa, and PhaC2=60.3 kDa.

FIG. 9 presents the kinetic data for wild type BktB thiolase and M158 BktB variant thiolase, in both condensation direction with acetyl-CoA, and thiolytic direction with acetoacetyl-CoA and 3-oxohexanoyl-CoA substrates. Curve fits shown are fit to the standard Michaelis-Menten equation.

FIG. 10A shows a model of the minimum energy structure of the active site of the wild type C. necator BktB thiolase with acetyl-CoA bound as priming acyl-CoA (Bind 1). All residues shown as ball and stick models are included in the mobile region of the conformational search.

FIG. 10B shows a model of the minimum energy structure of the active site of the M158A BktB thiolase with acetyl-CoA bound as priming acyl-CoA (Bind 1). Note that ΔΔE_{Bind A}^{Mut-WT} for Bind 1 is computed as the difference in binding energies between FIGS. 10A and 10B. All residues shown as ball and stick models are included in the mobile region of the conformational search.

FIG. 10C shows a model of the minimum energy structure of the active site of wild type C. necator BktB thiolase with butyryl-CoA bound as the priming acyl-CoA (Bind 1). Note that residues M158 and M290 adopt conformations different from the wild type structure with acetyl-CoA bound as priming acyl-CoA in FIG. 10A. All residues shown as ball and stick models are included in the mobile region of the conformational search.

FIG. 10D shows a model of the minimum energy structure of the active site of the M158A BktB thiolase with butyryl-CoA bound as priming acyl-CoA (Bind 1). Note that ΔΔE_{Bind B}^{Mut-WT} for Bind 1 is computed as the difference in binding energies between FIGS. 10C and 10D. All residues shown as ball and stick models are included in the mobile region of the conformational search.

FIG. 11A shows a model of the minimum energy structure of the active site of wild type C. necator BktB thiolase with acetyl-CoA bound as the extending acyl-CoA and C90 acetylated (Bind 2). All residues shown as ball and stick models are included in the mobile region of the conformational search.

FIG. 11B shows a model of the minimum energy structure of the active site of the M158A BktB thiolase with acetyl-CoA bound as the extending acyl-CoA and C90 acetylated (Bind 2). Note that ΔΔE_{Bind A}^{Mut-WT} for Bind 2 is computed as the difference in binding energies between FIGS. 11A and 11B. All residues shown as ball and stick models are included in the mobile region of the conformational search.

FIG. 11C shows a model of the minimum energy structure of the active site of wild type C. necator BktB thiolase with acetyl-CoA bound as the extending acyl-CoA and C90 butyrylated (Bind 2). All residues shown as ball and stick models are included in the mobile region of the conformational search.

FIG. 11D shows a model of the minimum energy structure of the active site of the M158A BktB thiolase with butyryl-CoA bound as priming acyl-CoA (Bind 1). Note that ΔΔE_{Bind B}^{Mut-WT} for Bind 1 is computed as the difference in binding energies between FIGS. 11C and 11D. All residues shown as ball and stick models are included in the mobile region of the conformational search.

FIG. 12A presents a schematic of the heterologous 3HA pathway that contains the two core reactions of PHA biosynthesis: those catalyzed by the thiolase and 3-ketoacyl-CoA reductase. The pathways may additionally include a CoA activator enzyme, enoyl-CoA reductases, hydratases, and thioesterases (as shown in FIG. 1A). This pathway may also operate in an iterative manner, with each cycle leading to the formation of compounds elongated by two carbons, as a result of the thiolase catalyzed condensation between acetyl-CoA and a different acyl-CoA (this is commonly known as reverse beta-oxidation). Generally, this pathway can be used to produce compounds that are C4-C10 in chain length.

FIG. 12B presents a schematic of optional extensions of the pathway shown in FIG. 12A by expressing enzymes that act on the acyl-CoA intermediates. Additional downstream enzymes may be overexpressed (e.g., thioesterases, enoyl-CoA reductases, and hydratase), and the pathway can be used to produce a multitude of molecules. Some of these enzymes include Car, a carboxylic acid reductase; a ω-hydroxylase such as AlkBGT from P. putida, and various alcohol and aldehyde dehydrogenases.

FIG. 12C shows examples of specific chemicals that may be produced utilizing the extended 3HA pathway: 2,3-DHBA, 3HBL, 4-phenyl-butyric acid, adipic acid, pentane ω-hydroxyoctanoic acid, and 4-methyl-pentanol.

FIG. 13 presents an alignment of the amino acid sequences of BktB, PhaA, Rru (Rru_A10274), and PhbA generated using Clustal Omega (Sievers et al, 2011). The amino acid residues of the catalytic triad are boxed. The amino acid sequence of each of the enzymes are provided as follows: BktB, SEQ ID NO: 2; PhaA, SEQ ID NO: 4; Rru_A0274, SEQ ID NO: 3; and PhbA, SEQ ID NO: 1.

FIG. 14A is a schematic of the pathway to product 4-methyl-pentanol in E. coli (adapted from Sheppard et al., 2014). Synthesis of 4-methyl-pentanol from glucose was implemented through a four-module pathway, which included valine synthesis (module 1), acetyl-CoA activation (module 2), reverse β-oxidation cycle (module 3), and acid reduction (module 4). An endogenous thioesterase was used to terminate the reverse β-oxidation cycle, shown as a dotted line. The pathway was tested using isobutyryl-CoA ligase (IbuA) from Rhodopseudomonas palustris and an alcohol dehydrogenase from Leifsonia sp. Strain 5749. One undesired byproduct of this pathway was butryrate resulting from condensation of two acetyl-CoA molecules and completion of the reverse β-oxidation pathway.

FIG. 14B shows the titers of 4-methyl-pentanol and butyrate titers achieved using BktB from C. necator. The wild-type (WT) BktB enzyme is shown in dark gray bars, and M158A BktB variant is shown in light gray bars.

### DETAILED DESCRIPTION OF INVENTION

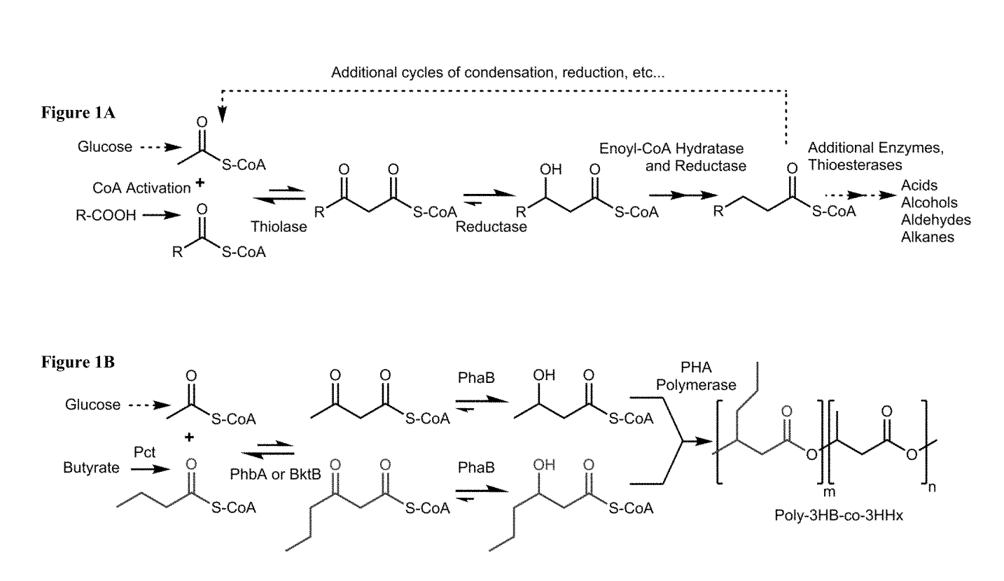

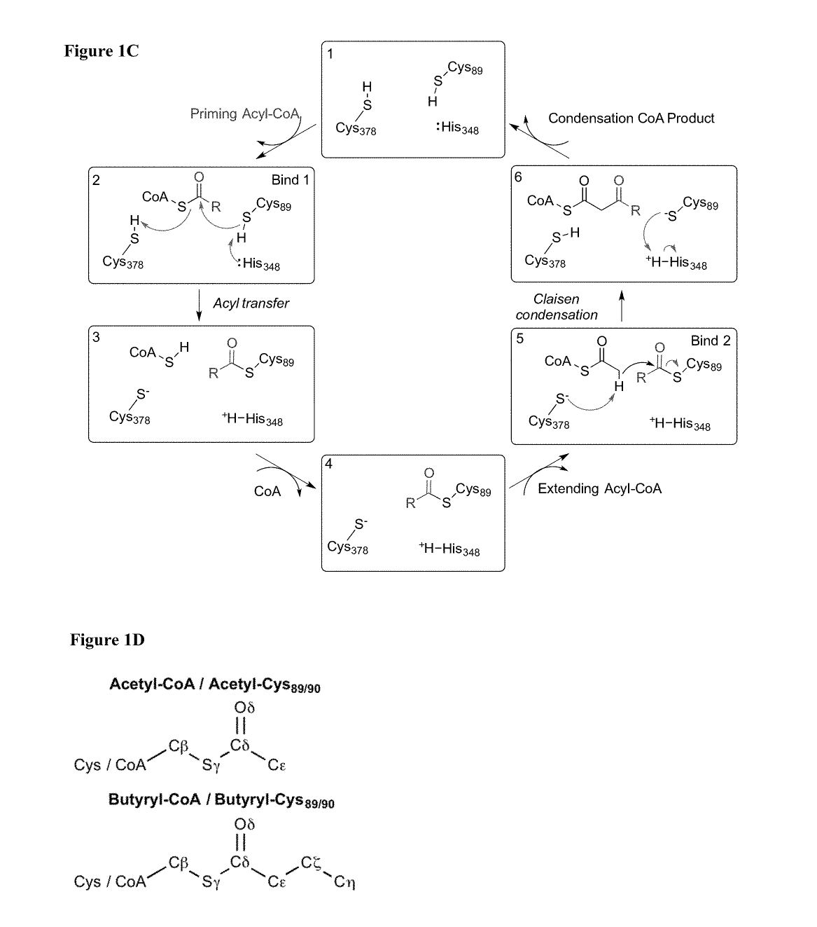

The 3-hydroxyacid (3HA) pathway (FIG. 1A), also referred to as the (partial) reverse β-oxidation or CoA-dependent chain elongation pathway, can allow for the synthesis of dozens of useful compounds of various chain lengths and functionalities, including acids, alcohols, alkanes and aldehydes, with applications in the pharmaceutical, polymer, flavor and fragrance industries (Clomburg et al., 2015; Kim et al., 2015; Sheppard et al., 2014; Tseng and Prather, 2012). This versatility is due to the promiscuous activities of pathway enzymes, which on the one hand makes the biological synthesis of these compounds possible, but on the other, results in a mixture of products at the end of the fermentation (Cheong et al., 2016; Clomburg et al., 2012). Separation and purification of desired production from undesired byproducts is both costly and time consuming. Thus, enzymes having increased substrate specificities are desired in order to maximize yields of desired products, to minimize downstream separation costs, avoid wasting cellular resources in the production of undesired products, and prevent the accumulation of potentially toxic intermediates. The lack of enzymes that are both highly active and highly specific toward the production of a particular product is a limitation in the construction of commercially feasible metabolic pathways.

Described herein are thiolase variants comprising amino acid substitutions at selected positions. In some embodiments, the thiolase variants have increased selectivity ratios and allow for the production of longer chain products (e.g., C6 products). Also described herein are cells that recombinantly express the thiolase variants and methods of producing desired products by culturing the cells described herein. In some embodiments, the cells also recombinantly express one or more additional enzymes involved in a biosynthetic pathway for the production of a desired product.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

Aspects of the present disclosure relate to thiolase variants having one or more amino acid substitutions at particular positions relative to a reference thiolase, which may be a wild type thiolase. A thiolase may also be referred to as an acetyl-coenzyme A acetyltransferase. As will be evident to one of ordinary skill in the art, a thiolase is an enzyme that is capable of catalyzing carbon-carbon bond formation/cleavage, in a cofactor independent manner. Thiolases generally belong to the enzyme classes EC:2.3.1.XX. Thiolases catalyze the condensation of a priming acyl-CoA and an extending acyl-CoA using a sequential bi bi ping-pong mechanism, such as the mechanism shown in FIG. 1C.

As used herein, a “variant” of a thiolase is a thiolase that contains one or more modifications to the primary amino acid sequence of the thiolase. Thiolase variants generally retain thiolase activity, e.g., the capability of catalyzing carbon-carbon bond formation/cleavage, in a cofactor independent manner. In some embodiments, a thiolase variant may have altered activity relative to the thiolase that is not a variant (e.g., a thiolase that does not contain the one or more modifications, e.g., wild type) or a reference thiolase. In some embodiments, the variant has increased activity relative to the thiolase that is not a variant (e.g., wild type) or a reference thiolase. In some embodiments, the variant has decreased activity relative to the thiolase that is not a variant (e.g., wild type) or a reference thiolase.

Generally, modifications which create a variant can be made to a polypeptide 1) to reduce or eliminate an activity of a polypeptide; 2) to enhance a property of a polypeptide; 3) to provide a novel activity or property to a polypeptide, such as addition of an antigenic epitope or addition of a detectable moiety; or 4) to provide equivalent or better binding between molecules (e.g., an enzymatic substrate). Modifications to a polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like.

Aspects of the present disclosure relate to thiolase variants that have been modified to increase the selectivity ratio of the thiolase. As used herein, the term “selectivity ratio” refers to the ratio of one or more desired products to one or more undesired product(s) (also referred to herein as byproducts). The activity of a thiolase can be promiscuous and typically results in the production of a mixture of products. The selectivity ratio may be increased by any of a number of methods, for example by increasing the production of the one or more desired products and/or by reducing the production of the one or more undesired products. In some embodiments, the thiolase variant results in an increase of the selectivity ratio by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more relative to the selectivity ratio of a thiolase that has not been modified (e.g., wild type). In some embodiments, the thiolase variant results in an increase in the production of one or more desired products by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more. In some embodiments, the thiolase variant results in an increase in the production of 3-oxo-hexanoyl-CoA, 3-hydroxy-hexanoic acid, or 3-hydroxy-hexanoate (3HHx) by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more. In some embodiments, the thiolase variant results in an increase in the production of 4-methyl pentanol by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more.

In some embodiments, the thiolase variant results in a decrease in the production of one or more undesired products by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more. In some embodiments, the thiolase variant results in a decrease in the production of acetoacetyl-CoA or 3-hydroxybutyric acid (3HB) by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more. In some embodiments, the thiolase variant results in a decrease in the production of butyrate by at least 2%, 3%, 4%, 5%, 6%, 7%, 8%, 9%, 10%, 15%, 20%, 25%, 30%, 40%, 45%, 50%, 60%, 70%, 80%, 90%, 100%, 200%, 300%, 400%, 500% or more.

In some embodiments, the thiolase variant results in a selectivity ratio of at least 1, meaning the cell produces an equal amount of the desired product and an undesired product. In some embodiments, the thiolase variant results in a selectivity ratio of at least 1.25, 1.5, 1.75, 2, 2.25. 2.5, 2.75, 3, 3.25, 3.5, 3.75, 4, 4.5, 5, 5.5, 6, 6.5, 7., 7.5, 8, 8.5, 9, 9.5, 10 or higher.

In some embodiments, the desired product is a longer chain product. As will be evident to one of ordinary skill in the art, a molecule having a carbon backbone may be classified based on the number of carbon molecules within the backbone. In some embodiments, the desired product is a C5, C6, C7, C8, C9, or C10 product. In some embodiments, the undesired product (byproduct) is a shorter chain product, relative to the desired product. In some embodiments, the selectivity ratio corresponds to the production of one or more C6 products relative to one or more C4 products. In some embodiments, the selectivity ratio corresponds to production of 3-hydroxyhexanoic acid (3HH) or 3HH-CoA relative to a C4 product. In some embodiments, the C4 product is 3-hydroxybutyryl-CoA (3HB-CoA). In some embodiments, the selectivity ratio corresponds to production of 4-methyl pentanol relative to butyrate.

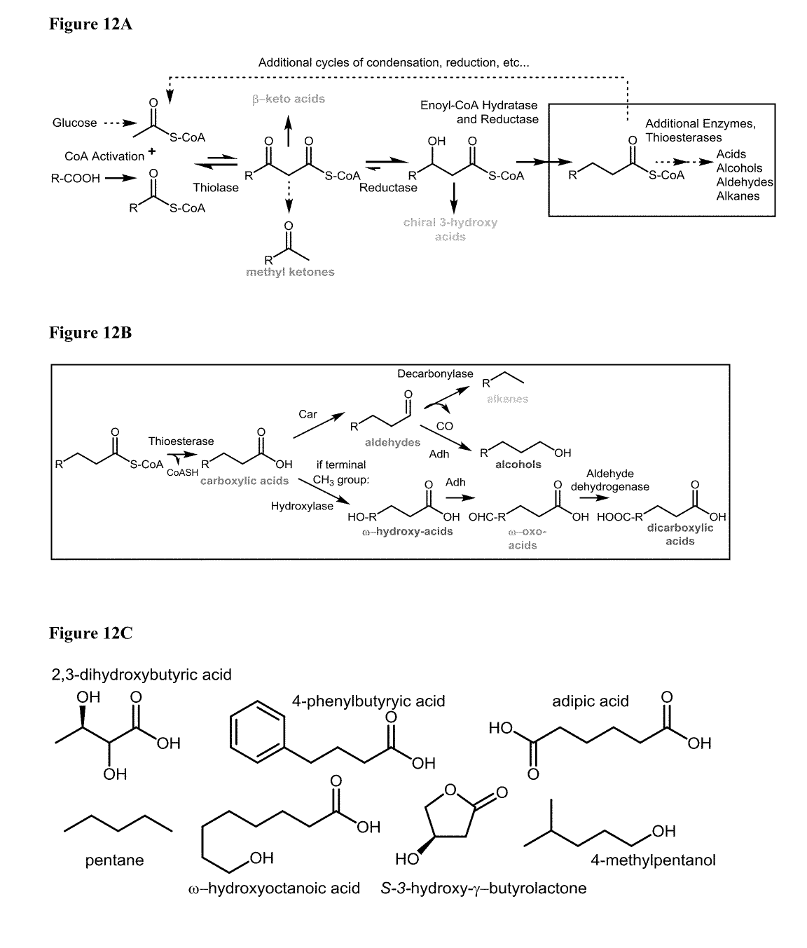

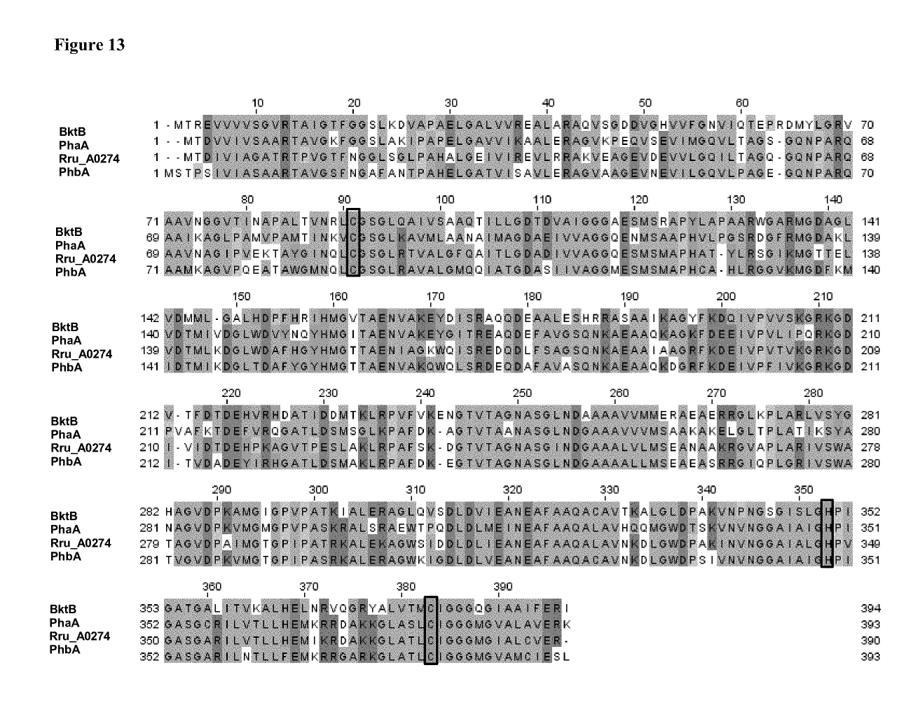

As described herein, thiolases and therefore the thiolase variants may be used in any of a variety of biosynthetic pathways. In some embodiments, the thiolase variants are used in a 3-hydroxyacid pathway. In some embodiments, the desired product is a 3-hydroxyacid, carboxylic acid, dicarboxylic acid, methyl ketone, hydroxyl-carboxylic acid, polyhydroxyalkanoate, keto-acid, aldehyde, alcohol, or alkane (FIGS. 12A and 12B). In some embodiments, the desired product is 2,3-dihydroxybutyric acid, 4-phenylbutyryic acid, adipic acid, pentane, ω-hydroxyoctanoic acid, S-3-hydroxy-γ-butyrolactone, or 4-methyl pentanol.

Positions in a thiolase amino acid sequence that can be modified (e.g., substituted) to produce a thiolase variants can be identified using any method known in the art. For example, as described herein, positions in PhbA from Z. ramigera were identified using available crystallographic information for the PhbA thiolase. In some embodiments, an amino acid substitution may allow for increased accessibility of a substrate (e.g., a longer substrate) to the active site of the thiolase. As used herein, the term “active site” refers to a region of the enzyme with which a substrate interacts. The amino acids that comprise the active site and amino acids surrounding the active site, including the functional groups of each of the amino acids, may contribute to the size, shape, and/or substrate accessibility of the active site. In some embodiments, the thiolase variant contains one or more modifications that are substitutions of a selected amino acid with an amino acid having a smaller functional group. In some embodiments, the thiolase variant contains one or more modifications that are substitutions of a selected amino acid with glycine, serine, or alanine.

This information can also be used to identify positions, e.g., corresponding positions, in other thiolases. As will be evident to one of ordinary skill in the art, an amino acid substitution at a position identified in one thiolase can also be made in the corresponding amino acid position of another thiolase. In such instances, one of the thiolases may be used as a reference thiolase. For example, as described herein, amino acid substitutions at position M157 of PhbA from Z. ramigera have been shown to increase the selectivity ratio of PhbA. Similar amino acid substitutions can be made at the corresponding position of another thiolase using PhbA as a reference. As will also be evident to one or ordinary skill in the art, the amino acid position number of a selected residue in a thiolase may have a different amino acid position number in another thiolase (e.g., a reference thiolase). For example, the methionine at position number 157 was selected for mutation in PhbA, however the corresponding methionine is at position 158 in the BktB amino acid sequence. Generally, one may identify corresponding positions in other thiolases using methods known in the art, for example by aligning the amino acid sequences of two or more thiolases, as shown in FIG. 13. Software programs and algorithms for aligning amino acid (or nucleotide) sequences are known in the art and readily available, e.g., Clustal Omega (Sievers et al. 2011).

The thiolase variants described herein may further contain one or more additional modifications, for example to specifically alter a feature of the polypeptide unrelated to its desired physiological activity. For example, cysteine residues can be substituted or deleted to prevent unwanted disulfide linkages. Similarly, certain amino acids can be changed to enhance expression of a polypeptide by eliminating proteolysis by proteases in an expression system (e.g., dibasic amino acid residues in yeast expression systems in which KEX2 protease activity is present).

Mutations of a nucleic acid which encodes a thiolase preferably preserve the amino acid reading frame of the coding sequence, and preferably do not create regions in the nucleic acid which are likely to hybridize to form secondary structures, such a hairpins or loops, which can be deleterious to expression of the variant thiolase.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon-optimization). The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of thiolase variant can be tested by cloning the gene encoding the thiolase variant into a bacterial or mammalian expression vector, introducing the vector into an appropriate host cell, expressing the thiolase variant, and testing for a functional capability of the thiolase, as disclosed herein.

The thiolase variants described herein contain an amino acid substitution of one or more positions corresponding to a reference thiolase. In some embodiments, the thiolase variant contains an amino acid substitution at 1, 2, 3, 4, 5, or more positions corresponding to a reference thiolase.

In some embodiments, the thiolase variant may also contain one or more amino acid substitutions that do not substantially affect the activity and/or structure of the thiolase. The skilled artisan will also realize that conservative amino acid substitutions may be made in the thiolase variant to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In general, it is preferred that fewer than all of the amino acids are changed when preparing variant polypeptides. In some embodiments, where particular amino acid residues are known to confer function, such amino acids will not be replaced, or alternatively, will be replaced by conservative amino acid substitutions. In some embodiments, preferably, 1, 2, 3, 4, 5, 6, 7, 8, 9, 10, 11, 12, 13, 14, 15, 16, 17, 18, 19, 20 residues can be changed when preparing variant polypeptides. It is generally preferred that the fewest number of substitutions that result in a desired activity are made. Thus, one method for generating variant polypeptides is to substitute all other amino acids for a particular single amino acid, then assay activity of the variant, then repeat the process with one or more of the polypeptides having the best activity.

Amino acid substitutions in the amino acid sequence of a polypeptide to produce a thiolase variant having a desired property and/or activity are made by alteration of a nucleic acid encoding the thiolase polypeptide. Similarly, conservative amino acid substitutions in the amino acid sequence of a polypeptide to produce functionally equivalent variants of the polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

Described herein are thiolase variants as well as additional enzymes that can be expressed in a cell, independently or in combination, for example in a biosynthetic pathway for the production of a desired product. As also described herein, the thiolase variants contain an amino acid substitution at one or more amino acid positions relative to a reference thiolase. The amino acid sequence or the nucleotide sequence encoding the amino acid sequence to the thiolase and/or any additional enzyme may be obtained from any source known in the art. In some embodiments, the thiolase and/or any additional enzymes are obtained from or derived from a microorganism, such as bacteria. As used herein, the phrase “derived from” refers to a sequence (amino acid sequence or nucleotide sequence) that is obtained from a source and then modified/mutated. In some embodiments, the thiolase or reference thiolase is PhbA, BktB, Rru or PhbA. In some embodiments, the thiolase or reference thiolase is PhbA from Zoogloea ramigera. In some embodiments, the thiolase or reference thiolase is BktB from Cupriavidus necator. In some embodiments, the thiolase or reference thiolase is Rru from Rhodospirillum rubrum. In some embodiments, the thiolase or reference thiolase is PhaA from Cupriavidus necator.

In some embodiments, the thiolase variant is derived from PhbA from Z. ramigera, and the thiolase variant contains an amino acid substitution at one or more positions corresponding to the amino acid sequence of PhbA thiolase (SEQ ID NO: 1) or another reference thiolase. In some embodiments, the thiolase variant is derived from PhbA and contains an amino acid sequence at one or more position corresponding to V57, Q87, L88, S91, L93, D146, L148, T149, D150, M157, M288, N316, I350, S353, L377, I379, or Q64 of PhbA from Z. ramigera (SEQ ID NO: 1). In some embodiments, the thiolase variant is derived from PhbA and contains an amino acid substitution at position 88 of leucine to serine (L88S). In some embodiments, the thiolase variant is derived from PhbA and contains an amino acid substitution at position 157 of methionine to alanine, glycine, or serine (M157A, M157G, M157S). In some embodiments, the thiolase variant is derived from PhbA and contains an amino acid substitution at position 388 of methionine to alanine, glycine, or serine (M388A, M388G, M388S).

Protein sequence of PhbA from Z. ramigera (NCBI
Accesson No: 1DLU_A)
(SEQ ID NO: 1)
MSTPSIVIASAARTAVGSFNGAFANTPAHELGATVISAVLERAGVAAGEV
NEVILGQVLPAGEGQNPARQAAMKAGVPQEATAWGMNQLCGSGLRAVALG
MQQIATGDASIIVAGGMESMSMAPHCAHLRAGGVKMGDFKMIDTMIKDGL
TDAFYGYHMGTTAENVAKQWQLSRDEQDAFAVASQNKAEAAQKDGRFKDE
IVPFIVKGRKGDITVDADEYIRHGATLDSMAKLRPAFDKEGTVTAGNASG
LNDGAAAALLMSEAEASRRGIQPLGRIVSWATVGVDPKVMGTGPIPASRK
ALERAGWKIGDLDLVEANEAFAAQACAVNKDLGWDPSIVNVNGGAIAIGH
PIGASGARILNTLLFEMKRRGARKGLATLCIGGGMGVAMCIESL

In some embodiments, the thiolase variant is derived from BktB from C. necator, and the thiolase variant contains an amino acid substitution at one or more positions corresponding to the amino acid sequence of BktB thiolase (SEQ ID NO: 2) or another reference thiolase. In some embodiments, the thiolase variant is derived from BktB and contains an amino acid sequence at one or more position corresponding to V57, R88, L89, S92, L94, A148, L149, H150, D151, M158, M290, N318, A320, F321, I352, T355, M379, I381, I387 or Y66 of BktB from C. necator (SEQ ID NO: 2). In some embodiments, the thiolase variant is derived from BktB and contains an amino acid substitution at position 158 of methionine to alanine, glycine, or serine (M158A, M158G, M158S). In some embodiments, the thiolase variant is derived from BktB and contains an amino acid substitution at position 290 of methionine to glycine or serine (M290G, M290S).

Protein sequence ofBktB from C. necator (UniProt
ID No: Q0KBP1.1)
(SEQ ID NO: 2)
MTREVVVVSGVRTAIGTFGGSLKDVAPAELGALVVREALARAQVSGDDVG
HVVFGNVIQTEPRDMYLGRVAAVNGGVTINAPALTVNRLCGSGLQAIVSA
AQTILLGDTDVAIGGGAESMSRAPYLAPAARWGARMGDAGLVDMMLGALH
DPFHRIHMGVTAENVAKEYDISRAQQDEAALESHRRASAAIKAGYFKDQI
VPVVSKGRKGDVTFDTDEHVRHDATIDDMTKLRPVFVKENGTVTAGNASG
LNDAAAAVVMMERAEAERRGLKPLARLVSYGHAGVDPKAMGIGPVPATKI
ALERAGLQVSDLDVIEANEAFAAQACAVTKALGLDPAKVNPNGSGISLGH
PIGATGALITVKALHELNRVQGRYALVTMCIGGGQGIAAIFERI

In some embodiments, the thiolase variant is derived from Rru from Rhodospirillum rubrum, and the thiolase variant contains an amino acid substitution at one or more positions corresponding to the amino acid sequence of Rru thiolase (SEQ ID NO: 3) or another reference thiolase. In some embodiments, the thiolase variant is derived from Rru and contains an amino acid sequence at one or more position corresponding to M156 or M287. In some embodiments, the thiolase variant is derived from Rru and contains an amino acid substitution at position 156 of methionine to alanine, glycine, or serine (M156A, M156G, M156S). In some embodiments, the thiolase variant is derived from Rru and contains an amino acid substitution at position 287 of methionine to alanine, glycine, or serine (M287A, M287G, M287S).

Protein sequence of Rru from Rhodospirillum rubrum
(NCBI Accession No: WP_011388027.1)
(SEQ ID NO: 3)
MTDIVIAGATRTPVGTFNGGLSGLPAHALGEIVIREVLRRAKVEAGEVDE
VVLGQILTAGQGQNPARQAAVNAGIPVEKTAYGINQLCGSGLRTVALGFQ
AITLGDADIVVAGGQESMSMAPHATYLRSGIKMGTTELVDTMLKDGLWDA
FHGYHMGTTAENIAGKWQISREDQDLFSAGSQNKAEAAIAAGRFKDEIVP
VTVKGRKGDIVIDTDEHPKAGVTPESLAKLRPAFSKDGTVTAGNASGIND
GAAALVLMSEANAAKRGVAPLARIVSWATAGVDPAIMGTGPIPATRKALE
KAGWSIDDLDLIEANEAFAAQALAVNKDLGWDPAKINVNGGAIALGHPVG
ASGARILVTLLHEMIKRDAKKGLATLCIGGGMGIALCVER

In some embodiments, the thiolase variant is derived from PhaA from C. necator, and the thiolase variant contains an amino acid substitution at one or more positions corresponding to the amino acid sequence of PhaA thiolase (SEQ ID NO: 4) or another reference thiolase. In some embodiments, the thiolase variant is derived from PhaA and contains an amino acid sequence at one or more position corresponding to M157 or M289. In some embodiments, the thiolase variant is derived from PhaA and contains an amino acid substitution at position 157 of methionine to alanine, glycine, or serine (M157A, M157G, M157S). In some embodiments, the thiolase variant is derived from PhaA and contains an amino acid substitution at position 289 of methionine to alanine, glycine, or serine (M289A, M289G, M289S).

Protein sequence ofPhaA from C. necator
(UniProtNo: P14611.1)
(SEQ ID NO: 4)
MTDVVIVSAARTAVGKFGGSLAKIPAPELGAVVIKAALERAGVKPEQVSE
VIMGQVLTAGSGQNPARQAAIKAGLPAMVPAMTINKVCGSGLKAVMLAAN
AIMAGDAEIVVAGGQENMSAAPHVLPGSRDGFRMGDAKLVDTMIVDGLWD
VYNQYHMGITAENVAKEYGITREAQDEFAVGSQNKAEAAQKAGKFDEEIV
PVLIPQRKGDPVAFKTDEFVRQGATLDSMSGLKPAFDKAGTVTAANASGL
NDGAAAVVVMSAAKAKELGLTPLATIKSYANAGVDPKVMGMGPVPASKRA
LSRAEWTPQDLDLMEINEAFAAQALAVHQQMGWDTSKVNVNGGAIAIGHP
IGASGCRILVTLLHEMKRRDAKKGLASLCIGGGMGVALAVERK

As one of ordinary skill in the art would be aware, homologous genes for these enzymes or any of the other enzymes described herein could be obtained from other species and could be identified by homology searches, for example through a protein BLAST search, available at the National Center for Biotechnology Information (NCBI) internet site (ncbi.nlm.nih.gov). By aligning the amino acid sequence of an enzyme with one or more reference thiolase and/or by comparing the secondary or tertiary structure of a similar or homologous enzyme with one or more reference thiolase, one can determine corresponding amino acid residues in similar or homologous enzymes and can determine amino acid residues for mutation in the similar or homologous enzyme.

Genes associated with the invention can be PCR amplified from DNA from any source of DNA which contains the given gene. In some embodiments, genes associated with the invention are synthetic. Any means of obtaining a gene encoding the enzymes associated with the invention are compatible with the instant invention.

The disclosure provided herein involves recombinant expression of genes encoding enzymes discussed above, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

Additional thiolases may be identified for which the methods and substitution mutations described herein may be applied. In some embodiments, the methods described herein and/or corresponding mutations described herein may be applied to a thiolase if the thiolase has at least 40% amino acid sequence identity to any of the thiolases described herein (e.g., SEQ ID NOs: 1-4). In some embodiments, the methods described herein and/or corresponding mutations described herein are applied to a thiolase if the thiolase has at least 45%, 50%, 55%, 60%, 65%, 70%, 75%, 80%, 85%, 90%, 95% or more amino acid sequence identity to one of the thiolases described herein.

In some embodiments, a thiolase may have a region of higher amino acid sequence identity with any of the thiolases described herein, as compared to the amino acid sequence identity over the full length of the thiolases. In some embodiments, the thiolase has higher amino acid sequence identity within the active site region with any of the thiolases described herein as compared to the amino acid sequence identity over the full length of the thiolases. In some embodiments, the methods and substitution mutations described herein may be applied to a thiolase if the region within 10 angstroms of the active site has greater than 75% amino acid identity to the corresponding region of any of the thiolases described herein. In some embodiments, the region of a thiolase that is within 10 angstroms of the active site has greater than 80%, 85%, 90%, 95% or more amino acid sequence identity to the corresponding region of any of the thiolases described herein. In some embodiments, the methods and substitution mutations described herein may be applied to a thiolase if the region within less than 10 angstroms of the active site has greater than 75% amino acid identity to the corresponding region of any of the thiolases described herein. In some embodiments, the region of a thiolase that is within 2, 3, 4, 5, 6, 7, 8, or 9 angstroms of the active site has greater than 75% amino acid identity to the corresponding region of any of the thiolases described herein. Methods for determining the three dimensional structure of an enzyme or modeling the three dimensional structure of an enzyme to determine regions within a particular distance of one another will be known to one of ordinary skill in the art.

Homology and/or identity can be calculated using various, publicly available software tools developed by NCBI (Bethesda, Md.) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

Aspects of the present disclosure relate to thiolase variants that can be used, for example as part of a biosynthetic pathway, for the production of desired products. In some embodiments, the thiolase variant is used in a 3-hydroxyacid pathway. In general, the 3HA pathway involves several core enzymes and can be modified to produce a variety of products (e.g., FIG. 1A). In some embodiments, a cell that recombinantly expresses a thiolase variant also expresses one or more additional enzymes. In some embodiments, the cell that recombinantly expresses a thiolase variant also expresses a Coenzyme A (CoA)-activating enzyme. In some embodiments, the CoA-activating enzyme is Pct, e.g., Pct from Megaphaera elsdenii. In some embodiments, the CoA-activating enzyme is IbuA, e.g., IbuA from Rhodopseudomonas palustris. In some embodiments, the cell that recombinantly expresses a thiolase variant also expresses a reductase. In some embodiments, the reductase is a PhaB, e.g., PhaB from Cupriavidus necator. In some embodiments, the cell that recombinantly expresses a thiolase variant also expresses a reductase. In some embodiments, the cell that recombinantly expresses a thiolase variant also expresses a thioesterase. In some embodiments, the thioesterase is TesB, e.g., TesB from E. coli.

In some embodiments, the cell that recombinantly expresses the thiolase variant also expresses one or more additional enzymes to produce a desired product. In some embodiments, the cell also expresses an enoyl-CoA reductase and/or a enoyl-CoA dehydratase. In some embodiments, the enoyl-CoA reductase is Ter, e.g., Ter from Treponema denticola. In some embodiments, the enoyl-CoA dehydratase is PhaJ4b, e.g., PhaJ4b from Cupriavidus necator. In some embodiments, the cell that recombinantly expresses the thiolase variant also expresses a poly-3-hydroxyalkanoate (PHA) polymerase. In some embodiments, the PHA polymerase is PhaC2, e.g., PhaC2 from Rhodococcus aetherivorans.

FIG. 12B shows several additional examples of enzymes that may also be expressed to produce desired products. In some embodiments, the cell that recombinantly expresses the thiolase variant also expresses an alcohol dehydrogenase and/or a aldehyde dehydrogenase. In some embodiments, the alcohol dehydrogenase is Adh, e.g., Adh from Leifsonia sp. In some embodiments, the cell that recombinantly expresses the thiolase variant also expresses a carboxylic acid reductase. In some embodiments, the carboxylic acid reductase is Car, e.g., Car from Nocardia iowensis. In some embodiments, the cell that recombinantly expresses the thiolase variant also expresses a hydroxylase, such as a ω-hydroxylase. In some embodiments, the ω-hydroxylase is A1kBGT, e.g., A1kBGT from P. putida.

The invention encompasses any type of cell that recombinantly expresses genes associated with the invention, including prokaryotic and eukaryotic cells. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In other embodiments the cell is a fungal cell such as yeast cells, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. Preferably the yeast strain is a S. cerevisiae strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments, the cell is an algal cell, a plant cell, an insect cell, an animal cell, or a mammalian cell.

It should be appreciated that some cells compatible with the invention may express an endogenous copy of one or more of the genes associated with the invention as well as a recombinant copy. In some embodiments if a cell has an endogenous copy of one or more of the genes associated with the invention then the methods will not necessarily require adding a recombinant copy of the gene(s) that are endogenously expressed. In some embodiments, the cell recombinantly expresses a thiolase variant, as described herein, and may endogenously express one or more enzymes from the pathways described herein. In some embodiments, the cell recombinantly expresses a thiolase variant and may recombinantly express one or more other enzymes from the pathways described herein.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for example for production of C6 products, such as 3HH-CoA, is demonstrated in the Examples section using E. coli. As one of ordinary skill in the art would appreciate, any of the enzymes described herein, including thiolase variants, can also be expressed in other bacterial cells, archaeal cells, fungi (including yeast cells), animal cells, mammalian cells, plant cells, etc.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments one or more genes associated with the invention is expressed recombinantly in a bacterial cell. Bacterial cells according to the invention can be cultured in media of any type (rich or minimal) and any composition. As would be understood by one of ordinary skill in the art, routine optimization would allow for use of a variety of types of media. The selected medium can be supplemented with various additional components. Some non-limiting examples of supplemental components include glucose, antibiotics, IPTG for gene induction, ATCC Trace Mineral Supplement, glycolate, butyrate, and propionate. Similarly, other aspects of the medium, and growth conditions of the cells of the invention may be optimized through routine experimentation. For example, pH and temperature are non-limiting examples of factors which can be optimized. In some embodiments, factors such as choice of media, media supplements, and temperature can influence production levels of a desired product, such as a C6 product. In some embodiments the concentration and amount of a supplemental component may be optimized. In some embodiments, how often the media is supplemented with one or more supplemental components, and the amount of time that the media is cultured before harvesting the desired product is optimized.

According to aspects of the invention, high titers of a desired product are produced through the recombinant expression of genes associated with the invention, in a cell. As used herein, “high titer” refers to a titer in the milligrams per liter (mg L⁻¹) scale. In some embodiments, the supernatant of the cell culture contains a high titer of the desired product, which may be further purified or isolated from the supernatant or cell culture. The titer produced for a given product will be influenced by multiple factors including the choice of media.

In some embodiments, the titer of the desired product is at least 10 mg L⁻¹. For example, the titer can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more than 300 mg L⁻¹. In some embodiments, the titer of the desired product is as least 0.1 g L¹. For example, the titer of the desired product can be at least 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50 g L⁻¹.

In some embodiments, the titer of the desired product is less than 10 mg L⁻¹. For example, the titer can be approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8 or 9.9 mg L⁻¹. As will be understood by one of skill in the art, the cell culture used to culture any of the cells described herein and/or the supernatant obtained from the cell culture may contain any of the indicated titers.

Aspects of the present disclosure relate to reducing the production of undesired products (e.g., byproducts) during biosynthesis of the desired product. In some embodiments, the thiolase variants described herein reduce the production of an undesired product by about 5%, 10%, 15%, 20%, 25%, 30%, 35%, 40%, 45%, 50%, 55%, 60%, 65%, 70%, 75%, 80%, 85%, 90%, 95% or more relative to production of the undesired product using a thiolase that is not a variant (e.g., wild type).

Any liquid culture medium that allows growth of the cells can be compatible with aspects of the invention. In some embodiments the growth medium is Luria Broth (LB). In some embodiments, of the methods associated with the invention, the growth medium is supplemented with glucose. In some embodiments, the growth medium is supplemented with 1% glucose. In some embodiments, the growth medium is supplemented with butyrate. In certain embodiments, the growth medium is supplemented with approximately 15 mM butyrate. In some embodiments, both glucose and butyrate are used to supplement the growth medium.

The liquid cultures used to grow cells associated with the invention can be housed in any of the culture vessels known and used in the art. In some embodiments, large scale production in an aerated reaction vessel such as a stirred tank reactor can be used to produce large quantities of products.

Aspects of the invention include strategies to optimize production of a desired product from a cell. Optimized production of a desired product refers to producing a higher amount of the desired product following pursuit of an optimization strategy than would be achieved in the absence of such a strategy. Also within the scope of optimizing production of a desired product is reducing the production of an undesired product (e.g., a byproduct) or increasing the ratio of a desired product to an undesired product (e.g., the selectivity ratio) produced by the cell. One strategy for optimization is to increase expression levels of one or more genes associated with the invention through selection of appropriate promoters and/or ribosome binding sites. In some embodiments, this may include the selection of high-copy number plasmids, or low or medium-copy number plasmids. In some embodiments, the plasmid is medium-copy number plasmid such as pETDuet. The step of transcription termination can also be targeted for regulation of gene expression, through the introduction or elimination of structure such as stem-loops.

In some embodiments, it may be advantageous to use a cell that has been optimized for production of a desired product. In some embodiments, screening for one or more mutations that lead to enhanced production of the desired product (or reduced production of an undesired product) may be conducted through random mutagenesis, or through screening of known mutations. In some embodiments, shotgun cloning of genomic fragments can be used to identify genomic regions that lead to an increase in production of a desired product, through screening cells or organisms that have these fragments for production of the desired product. In some cases on or more mutation may be combined in the same cell or organism.

Optimization of production of a desired product can involve optimizing selection of cells for expression of recombinant pathways described herein. In some embodiments, use of a bacterial strain that is close to wild-type, meaning a strain that has not been substantially genetically modified, may lead to increased titers of the desired product.

Optimization of protein expression may also require in some embodiments that a gene encoding an enzyme be modified before being introduced into a cell such through codon optimization for expression in a bacterial cell. Codon usages for a variety of organisms can be accessed in the Codon Usage Database (kasusa.or.jp/codon/).

As described herein, the thiolase variants contain an amino acid substitution at one or more positions of the thiolase enzyme. However, in some embodiments, the expression and/or activity of any one or more of the additional enzymes expressed for the production of a desired product may also be modified. In some embodiments, one or more of the thiolases and/or one or more of the additional enzymes are subjected to a protein engineering approach, which could include determining the 3D structure of an enzyme or constructing a 3D homology model for the enzyme based on the structure of a related protein. Based on 3D models, mutations in an enzyme can be constructed and incorporated into a cell or organism, which could then be screened for an increase production of a desired product. In some embodiments, production of a desired product in a cell could be increased through manipulation of enzymes that act in the same pathway as the enzymes associated with the invention. For example in some embodiments it may be advantageous to increase expression of an enzyme or other factor that acts upstream of a target enzyme such as an enzyme associated with the invention. This could be achieved by over-expression of the upstream factor using any standard method.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLE

### Example 1: Rational Design of Thiolase Variants with Substrate Specificity

In the 3HA pathway, the thiolase enzyme sets the chain length upon which the other downstream enzymes act. Described herein are thiolase variants that have increased selectivity with high catalytic activity towards the synthesis of longer chain products. Previously, the 3HA pathway has been used for production of 3-hydroxy-valeric acid (3HV) and 3-hydroxy-hexanoic acid (3HH). While 100% conversion of the fed propionate precursor for the synthesis of 3HV has been achieved, less than 1% of the fed butyrate was converted to 3HH, indicating poor specificity and activity of the pathway enzymes towards the longer chain substrates (Martin et al., 2013).

Here, the initial focus was on achieving selective production of the longer chain C6 product, 3-hydroxyhexanoyl-CoA (3HH-CoA), relative to the C4, 3-hydroxybutyryl-CoA (3HB-CoA). Formation of 3HH-CoA results from the initial thiolase catalyzed condensation of a priming butyryl-CoA and extending acetyl-CoA, and subsequent action of a reductase on the condensation product (FIG. 1A). In contrast, 3HB-CoA is formed by the condensation of two acetyl-CoA substrates followed by reduction (FIG. 1B). Thus, it was sought to increase the thiolase selectivity ratio, which in this embodiment refers to the ratio of C6 product formed relative to the C4 product. Ideally, the ratio of the C6 product to the C4 product would be measured at the end of the thiolase catalyzed reaction (i.e., 3-oxo-hexanoyl-CoA to acetoacetyl-CoA), but the thermodynamics of this reaction require coupling to a downstream enzyme to enable product formation. Thus, as a proxy, the formation of free 3HH and 3HB was used, as well as PHAs containing those monomers, which are derived from 3HH-CoA and 3HB-CoA, the condensation products after the reductase step.

To arrive at a more selective thiolase, two general approaches could be pursued: bioprospecting or protein engineering, the latter including both rational engineering and directed evolution approaches. The decision to undertake a given approach hinges on the amount of information available at the outset of the study, as well as the throughput of the method that will be used to assay the resulting sequence space (Hicks and Prather, 2014). Bioprospecting for more selective thiolases presents several difficulties because very few have been extensively characterized and employed in heterologous pathways despite the fact that thiolase enzymes are ubiquitous in nature, being central to many biochemical processes such as fatty acid biosynthesis and degradation, PHA biosynthesis, and the Clostridial ABE fermentative pathway (Haapalainen et al., 2006). Specifically, the BktB thiolase from Cupravidus necator (formerly Ralstonia eutropha) has been used in the biosynthesis of hydroxyacids and alcohols from C4-C10 in chain length (Cheong et al., 2016; Martin et al., 2013; Sheppard et al., 2014). Interestingly, C. necator also has 14 other genes in its genome annotated as putative thiolases, however only BktB and one other thiolase, PhaA, have been characterized and explored for metabolic engineering purposes (Reinecke and Steinbüchel, 2008). The catalytic activity of BktB or other thiolases towards >C6 substrates and products has not been studied due to several inherent challenges, and due to the commercial unavailability of required acyl-CoA substrates.

Attempts at rational engineering of the thiolases have been limited due to the lack of in vitro data. A selection platform or a high-throughput screen would allow for one to assay a large number of variants. However, such methods have not been available for the thiolase—the reasons for which become apparent upon examination of the mechanism of the Claisen condensation reaction catalyzed by the thiolase.

Thiolases catalyze the condensation of a priming acyl-CoA and an extending acyl-CoA using a sequential bi bi ping-pong mechanism (FIG. 1C). The condensation of butyryl-CoA and acetyl-CoA to form 3-oxo-hexanoyl-CoA with high specificity was of interest; however, it was not possible to directly assay for this reaction for several reasons. First, for biosynthetic thiolases, such as BktB from C. necator and PhbA from Zoogloea ramigera, the condensation direction is thermodynamically unfavorable, requiring the condensation product to be reacted further in order to drive the reaction forward (Thompson et al., 1989). In this Example, the thiolase is coupled with a kinetically competent dehydrogenase enzyme. Reacting away CoASH, the other product of the condensation reaction is insufficient to drive the reaction forward because it is released in the first half-step of the overall condensation reaction mechanism. In addition, the self-condensation of two acetyl-CoAs will always occur with some frequency, biasing any measured reaction rate. However, the low yields and high cost of synthesis of these acyl-CoAs precluded the development of a high-throughput activity screen.

There are only two examples of attempts to engineer thiolases. The first attempt at thiolase engineering described in the literature used directed evolution to arrive at a variant that exhibited robust acetoacetyl-CoA product formation but lower sensitivity to inhibition by CoASH (Mann and Liitke-Eversloh, 2012). Another effort to engineer a thiolase to accommodate α-substituted acyl-CoAs relied on intuition guided rational mutagenesis of just one residue in close proximity of the active site but employed coenzyme-A analogs (Fage et al., 2015).

Here, limited by a low throughput in vivo assay, the extensive available crystallographic data was used to generate a computationally-driven, structure-guided rational engineering approach to engineer a biosynthetic thiolase for improved selectivity towards the synthesis of longer chain products. A theoretical framework for the design of ordered binding in a sequential bi bi ping-pong reaction is presented herein. This framework was initially applied to the biosynthetic thiolase PhbA from Z. ramigera for which there is ample crystallographic data but which exhibits low activity towards longer chain substrates, such as butyryl-CoA, to identify mutants which are predicted to exhibit higher selectivity ratios compared to the wild type thiolase. This same approach was then applied to the more active C. necator BktB thiolase. Mutations that were computationally predicted to improve the selectivity ratio of the thiolase were initially screened in vivo within the context of two heterologous pathways, free HA production and PHA biosynthesis, which employ different downstream enzymes (thioesterase vs. PHA polymerase).

As described herein, this process led to the identification of thiolase mutants with up to ten-fold increases in the selectivity ratio. In vitro characterization confirmed that one of the most selective mutants had 30-fold lower activity towards formation of the 3HB product, whereas the activity towards 3HH formation was comparable to wild type. This study also helped to expand the understanding of the sequence-structure-function relationship for the important thiolase class of enzymes, which represents a large conserved superfamily of enzymes central to many other biological pathways.

### Results

Computational Design of Mutants Predicted to Exhibit Increased Selectivity in Z. ramigera PhbA Thiolase

The structure of PhbA thiolase from Zoogloea ramigera has been investigated, with crystal structures representing each step of the catalytic cycle, for a total of 22 structures, including the following mutants: C89A, N316A/H/D, H348A/N, N316H-H348N and Q64A; (Kursula et al., 2002; Meriläinen et al., 2008; Meriläinen et al., 2009; Modis and Wierenga, 1999b; Modis and Wierenga, 2000). The structures of the C89A mutant with acetyl-CoA bound (1M3Z; 1.7 Å), the wild type thiolase with acetyl-CoA bound and C89 acetylated (1DM3; 2.0 Å), and unliganded wild type thiolase with C89 butyrylated (1M4T; 1.77 Å) provided a structural basis for examining acyl group specificity at each binding event (Kursula et al., 2002; Modis and Wierenga, 2000). Due to availability of this crystallographic data, PhbA was chosen as the starting point for structure-based design calculations. No published BktB crystal structures were available upon initiation of the work described herein.

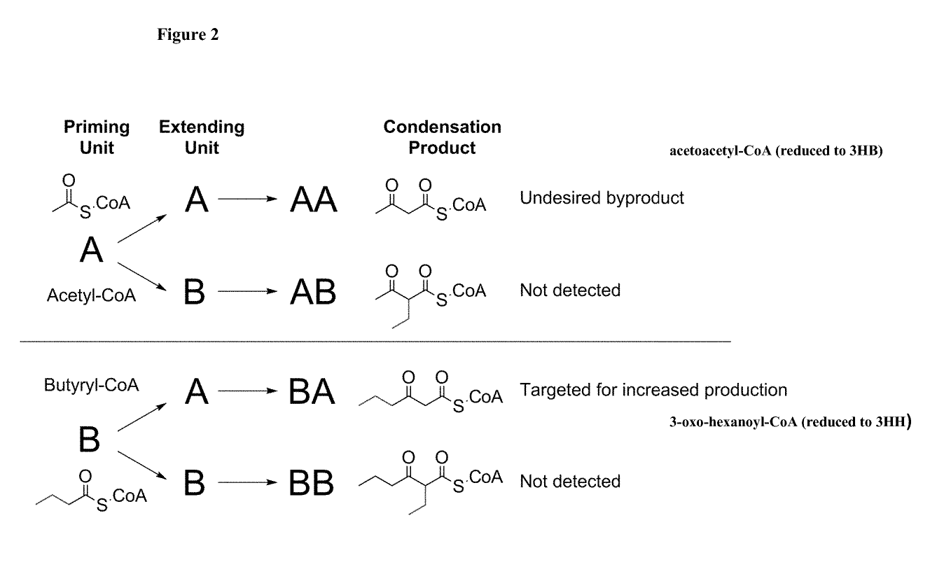

In the case of the 3HA pathway, improving the overall pathway selectivity ratio, e.g., the production of the longer chain C6, BA product relative to AA, using the nomenclature in FIG. 2 was of interest. At the thiolase level, this ratio could be improved either by increasing the formation of 3-oxohexanoyl-CoA (BA), by decreasing the formation of acetoacetyl-CoA (AA), or a combination of the two approaches. Without wishing to be bound by any particular theory, there are several possible steps in the thiolase catalytic cycle where BA production might be limited compared to AA production. For example, steric constraints might lead the thiolase active site to preferentially accommodate acetyl-CoA relative to butyryl-CoA during the priming CoA binding step (“Bind 1” in FIG. 1C). Similarly, steric constraints could also prevent the thiolase active site from accommodating butyrylated-C89 relative to acetylated-C89 in a conformation favorable to nucleophilic attack by the acetyl carbon of acetyl-CoA (“Bind 2” in FIG. 1C). Effectively, the butyryl group must be accommodated in at least two orientations in the active site: on the bound priming butyryl-CoA, and on the butyrylated catalytic C89.

While it is possible that one of the catalytic steps (e.g., proton abstraction, breakdown of acyl-enzyme intermediate) may limit BA production, crystallographic studies by Kursula et al (2002) suggest that butyrylation of C89 may inhibit catalytically productive binding of the extending acetyl-CoA. Kursula et al (2002) report that soaking experiments with butyryl-CoA and wildtype PhbA crystals result in butyrylation of C89 with no detectable CoA bound, indicating that butyryl-CoA is able to act as the priming acyl-CoA, but not as the extending acyl-CoA once the enzyme is butyrylated. Very low levels of 3HH formation were observed in vivo with wildtype PhbA, suggesting poor PhbA activity with butyryl-CoA as the priming acyl-CoA and acetyl-CoA as the extending acyl-CoA. On studies of ketosynthase domains in polyketide synthetases, which exhibit a similar bi bi ping-pong mechanism, it has been reported that the extending step is more often the bottleneck for acceptance of alternative substrates than the priming/acylation step (Jenner et al., 2015).

Superimposing the butyrylated C89 structure (which corresponds to step 4 of the catalytic cycle in FIG. 1C; PDB: 1M4T) upon the acetylated structure with acetyl-CoA bound as the extending acyl-CoA (representing step 5 in FIG. 1C; PDB: 1M3Z) reveals that the butyryl group of C89 lies directly over the sulfur atom of the bound extending acetyl-CoA, with the butyryl group pointing directly into the hydrophobic pocket formed by conserved active site residues M157 and M288, and the thioester oxygen pointing into the oxyanion hole formed by N(C89) and N(G380) (Kursula et al., 2002). It was sought to develop an in silico model that would allow prediction of the ability of mutations at these positions, as well as additional positions to allow the butyrylated C89 to take on a conformation more favorable to catalytically productive binding of acetyl-CoA as the extending acyl-CoA.

Rather than building models of the transition state for each step of the condensation reaction and optimizing the active site binding of the transition state associated with each catalytic step leading to BA production, the published crystal structures of acetyl-CoA bound as the priming acyl-CoA (1M3Z) and acetyl-CoA bound as the extending acyl-CoA with C89 acetylated (1DM3) were used. The crystal structures were assumed to represent catalytically productive binding modes at each step, and mutations which could accommodate a butyryl group in the appropriate place while keeping the rest of the crystal structure fixed outside of a defined radius (4.75 Å) of the residue to be mutated were identified (see “Computational Methodology” Section).

Although poor binding affinity of the extending acetyl-CoA due to the native conformation of butyryl-C89 may be a driver for poor BA production, the effect of active site mutations on the ability to accommodate butyryl-CoA as the priming acyl-CoA was also considered. If a thiolase mutant was able to accommodate the butyryl group in Bind 2, but as a result of the mutation was unable to accommodate the butyryl group in Bind 1, then this would likely lead to poor BA production. Although Kursula, et al (2002) observed that butyryl-CoA was capable of acting as the priming acyl-CoA with wild type PhbA, PhbA was reported to have a poor (mM) affinity for butyryl-CoA. Mutants were designed that did not further decrease this affinity.

Thus, design calculations were performed on conformations representing both Bind 1 and Bind 2. Structure-based design calculations were focused on identifying mutations with the potential to improve the energy of bound conformations leading to BA relative to those leading to AA, at either the first or second Michaelis complex (steps 2 and 5 in FIG. 1C, respectively).

Design calculations were performed as described in the Materials and Methods section herein below. Table I lists the PhbA mutants chosen for experimental testing along with their corresponding values of ΔΔE_{Bind B}^{Mut-WT}, ΔΔE_{Bind A}^{Mut-WT}, and ΔΔΔE_{Bind B-Bind A}^{Mut-WT} for both Bind 1 and Bind 2.

TABLE I
Energetic calculations of Z. ramigera PhbA variants selected for experimental testing

| | Bind 1^{(a)} | | | Bind 2^{(b)} | | |
|---|---|---|---|---|---|---|
| Mutant | ΔΔE_{Bind B}^{Mut-WT} | ΔΔE_{Bind A}^{Mut-WT} | ΔΔΔE_{Bind B-A}^{Mut-WT} | ΔΔE_{Bind B}^{Mut-WT} | ΔΔE_{Bind A}^{Mut-WT} | ΔΔΔE_{Bind B-A}^{Mut-WT} |
| L88S | −0.09 | 0.09* | −0.18 | −1.05 | 0.09* | −1.14 |
| L88A | 0.02* | 0.09* | −0.07 | −1.33 | 0.09* | −1.42 |
| L88G | 0.53* | 0.15* | 0.38* | −1.90 | −0.26 | −1.64 |
| M157S | −18.09 | 0.83* | −18.92 | −3.98 | 1.32* | −5.30 |
| M157A | −17.36 | 1.38* | −18.74 | −2.76 | 1.88* | −4.64 |
| M157G | −16.34 | 2.20* | −18.54 | −1.34 | 2.37* | −3.71 |
| M288S | 1.60* | 1.18* | 0.43* | −0.03 | 0.48* | −0.52 |
| M288A | 1.85* | 1.25* | 0.60* | 1.55* | 0.57* | 0.98* |
| M288G | 2.29* | 1.34* | 0.95* | 1.08* | 0.65* | 0.43* |
| L377S | −0.65 | 0.02* | −0.68 | 0.50 | 0.02* | 0.47* |
| L377A | −1.01 | 0.07* | −1.09 | −0.30 | 0.08* | −0.37 |
| L377G | −1.14 | 0.11* | −1.26 | 0.35* | 0.12* | 0.22* |

In the Bind 1^{(a)} column, ΔΔE_{Bind B}^{Mut-WT} is the difference in binding energies between mutant and wild type bound to butyryl-CoA with free C89; ΔΔE_{Bind A}^{Mut-WT} is the difference in binding energies between mutant and wild type bound to acetyl-CoA with free C89; and ΔΔΔE_{Bind B-A}^{Mut-WT} is the difference between ΔΔE_{Bind B}^{Mut-WT} and ΔΔE_{Bind A}^{Mut-WT}, corresponding to the differential specificities for mutant versus wild type for binding butyryl-CoA versus acetyl-CoA as the priming acyl-CoA.

In the Bind 2^{(b)} column, ΔΔE_{Bind B}^{Mut-WT} is the difference in binding energies between mutant and wild type bound to acetyl-CoA with butyrylated C89; ΔΔE_{Bind A}^{Mut-WT} is the difference in binding energies between mutant and wild type bound to acetyl-CoA with C89 acetylated; and ΔΔE_{Bind B-A}^{Mut-WT} is the difference between ΔΔE_{Bind B}^{Mut-WT} and ΔΔE_{Bind A}^{Mut-WT}, corresponding to the differential specificities for mutant versus wild type for binding acetyl-coA as the extending CoA with C89 either butyrylated or acetylated. Negative energies are indicated with a “*”, and positive energies are unmarked. All energies are reported in kcal/mol.

Mutants presented in Table I and chosen for experimental validation involve paring down of a bulky hydrophobic (L88, M157, M288, L377) residue to a smaller residue, such as serine, alanine or glycine. All mutants except M288A and M288G have negative values of ΔΔΔE_{Bind B-Bind A}^{Mut-WT} in Bind 1, Bind 2 or both Bind1 and Bind 2. All mutants chosen for experimental testing also have negative values of ΔΔE_{Bind B}^{Mut-WT} in either Bind 1 or Bind 2. All mutants also have positive values of ΔΔE_{Bind A}^{Mut-WT} Bind A in both steps, indicating decreased binding preference for accommodating for the acetyl group in both binding events.

Of all positions, M157 was judged the most promising candidate due to its negative values of ΔΔΔE_{Bind B-Band A}^{Mut-WT} in both binding events, the high magnitude of ΔΔΔE_{Bind B-Bind A}^{Mut-WT} relative to the other mutants, and the trend of ΔΔΔE_{Bind B-Bind A}^{Mut-WT} was exhibited for each of the similar mutations of M157S/A/G.

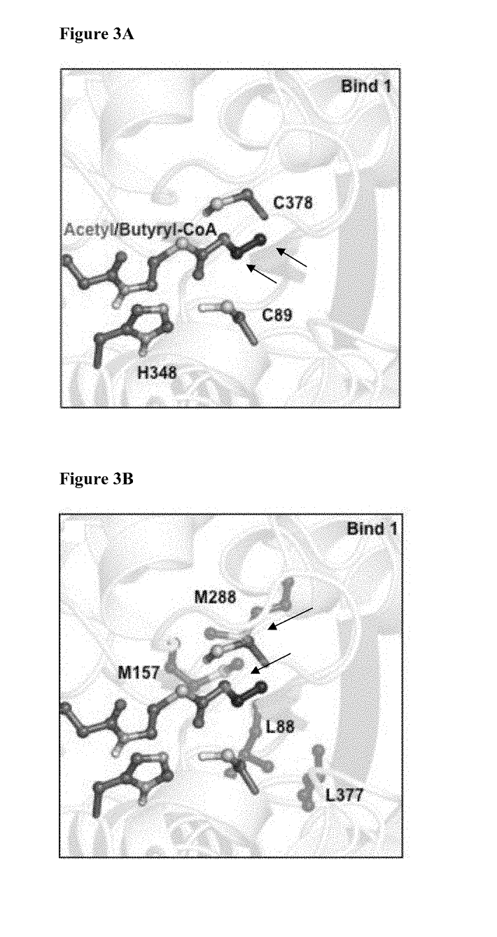

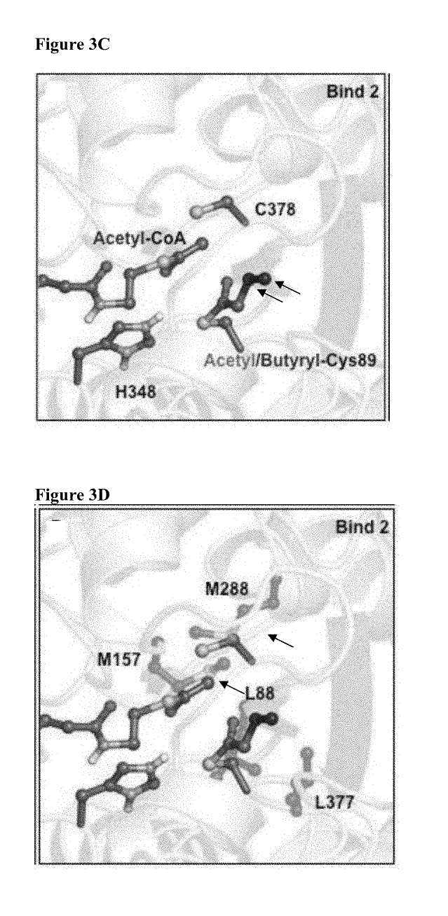

According to energetic calculations and upon inspection, M288S appeared to be a promising candidate for improving selectivity in Bind 2 but not M288A and M288G. M288A and M288G were also chosen for testing to account for the possibility that the model might not be able to accurately distinguish the small chemical differences between serine, alanine and glycine. FIGS. 3A-3D show the location of the residues chosen for PhbA mutagenesis relative to the active site catalytic residues in both the Bind 1 and Bind 2 orientations.

Based on the PhbA design calculations, the following 17 positions were identified to be mutated: V57, Q87, L88, S91, L93, D146, L148, T149, D150, M157, M288, N316, I350, S353, L377, I379 and Q64. Based on the BktB design calculations, the following 20 positions were identified to be mutated: V57, R88, L89, S92, L94, A148, L149, H150, D151, M158, M290, N318, A320, F321, I352, T355, M379, I381, I387 and Y66.

Initial Screening of Z. ramigera PhbA Mutants Identifies Several Improved Enzyme Variants

Z. ramigera PhbA thiolase mutants were initially assayed in vivo in the context of a previously established pathway for 3-hydroxyalkanoic acid (3HA) production (Martin et al., 2013b). This pathway consists of an activator enzyme (Pct from M. elsdenii), a thiolase (BktB from C. necator or PhbA from Z. ramigera), an NADPH dependent reductase (PhaB from C. necator), and a thioesterase (TesB from E. coli), which generates the final 3HA product. Specifically, when the cultures are supplied with butyrate and grown on glucose, the cells produce 3HB and 3HH. Examining the amount of 3HH produced relative to 3HB provides a measure of thiolase selectivity, which may be represented as the ratio of 3HH to 3HB.

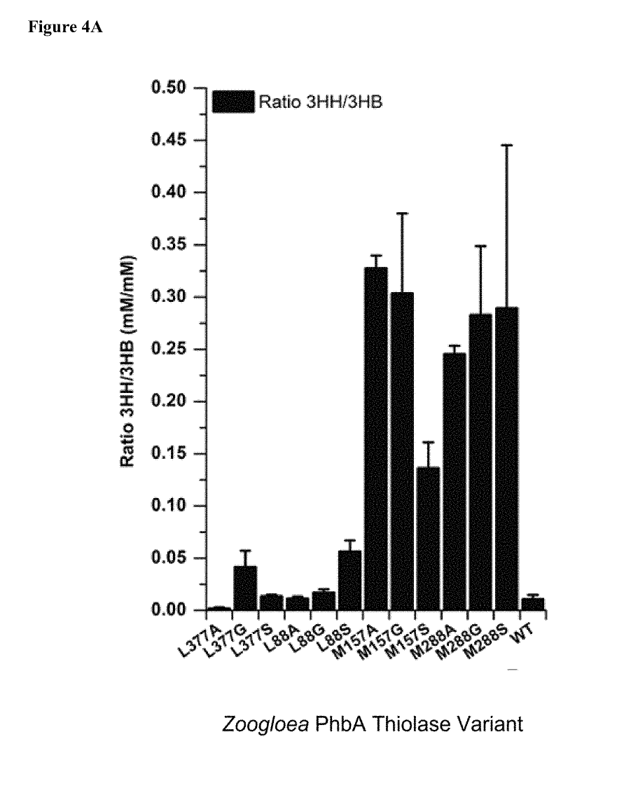

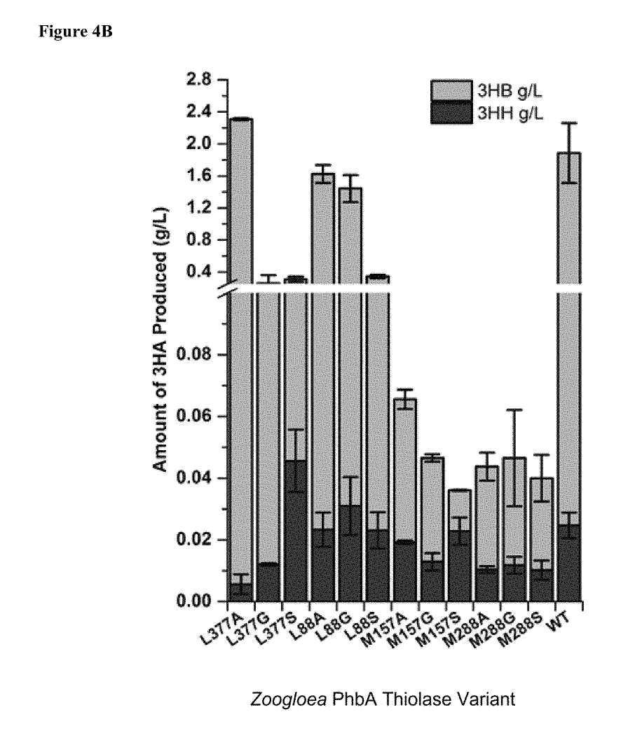

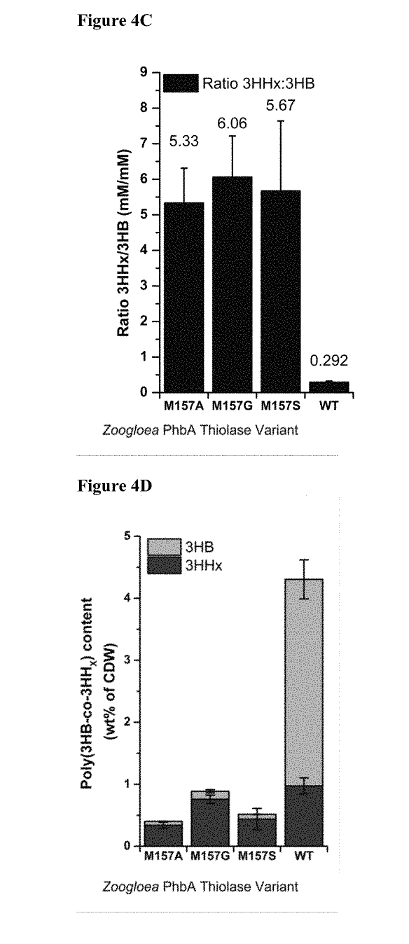

Of the twelve thiolase variants tested, several variants resulted in increased selectivity ratios in vivo (FIG. 4A). This higher selectivity ratio was mostly due to decreased production of 3HB by the pathway, and not increased 3HH titers (FIG. 4B). Specifically, five mutants: M157A/G and M288S/A/G resulted in an approximately 30-fold higher ratio of 3HH relative to the undesired 3HB by-product, with a roughly 80-fold decrease in their sum. The most selective mutants were further characterized. Because the extent to which the enzymes downstream of the thiolase could affect final product distribution was unknown, the mutants were also assayed within the context of another pathway. Thioesterases exhibit varying levels of activity towards different acyl-CoA substrates, depending on the carbon chain length and functional group of the substrate. The PHA biosynthesis pathway was thus subsequently used to screen the thiolase mutants as it is known that over 100 different 3HA monomers can be incorporated into PHAs, suggesting a broad substrate range for the PHA synthase (Agnew and Pfleger, 2013). The PhaC2 polymerase enzyme from R. aetherivorans 124 was selected for its ability to synthesize PHA polymers with large amounts of the longer chain C6 monomer, 3-hydroxyhexanoate, 3HHx (Budde et al., 2011). Using the polymerase as the terminal enzyme removes any possible limitation or specificity imposed by the thioesterase, providing further evidence for thiolase imposed selectivity on the distribution of observed products.

When the most selective PhbA thiolase variants were profiled using the PHA assay, M157 mutants resulted in an 18-fold higher 3HHx:3HB selectivity ratio (FIG. 4C). The resulting PHA polymers synthesized by M158A/G/S thiolase mutants contained 83-85 mol % of the 3HHx monomer, as compared to wild type, which only resulted in a 22 mol % of the 3HHx monomer (Table IV). To eliminate the possibility that native E. coli thiolases or reductases could influence final PHA composition, several control experiments were performed; no PHA accumulation was observed without plasmid-based overexpression of all four genes of the pathway (data not shown). These results were consistent with previous observations and support the initial hypothesis of these thiolases exhibiting reduced activity towards the condensation of two acetyl-CoAs, while maintaining similar or better activity towards the condensation of butyryl-CoA and acetyl-CoA compared to the wild type enzyme.

Validated Computational Approach Applied to More Active BktB Thiolase from C. necator

Having successfully identified mutants with increased C6/C4 (BA/AA) selectivity in the PhbA thiolase, the newly validated modeling framework was applied to identify mutants that might increase selectivity of the more active C. necator BktB thiolase. Although the BktB thiolase only exhibits 51% sequence identity with PhbA, the active site is highly similar, with 86% of the residues within 10 Å of the PhbA acetyl-CoA carbonyl center conserved between PhbA and BktB (Table II). Two unliganded crystal structures were available in the PDB for BktB (Fage et al., 2015; Kim et al., 2014b), and due to the active-site similarity, the Z. ramigera PhbA structures, 1M3Z and 1DM3 were used as templates to build structures of BktB with acetyl-CoA and butyryl-CoA bound.

TABLE II
Differences between PhbA and BktB active sites

| Distance | | | | | | |
|---|---|---|---|---|---|---|
| from | | | | | | |
| Acetyl- | | | | | | Cumulative |
| CoA | PhbA/ | | | | | BktB |
| Carbonyl | BktB | PhbA | PhbA | BktB | BktB | Residue |
| Center | Chain | ResID | Resname | ResID | Resname | Differences |
| 3.89 | A | 348 | H | 350 | H | 0 |
| 3.93 | A | 378 | C | 380 | C | 0 |
| 3.97 | A | 318 | A | 320 | A | 0 |
| 4.01 | A | 288 | M | 290 | M | 0 |
| 4.39 | A | 89 | C | 90 | C | 0 |
| 5.79 | A | 316 | N | 318 | N | 0 |
| 6.30 | A | 319 | F | 321 | F | 0 |
| 6.53 | A | 147 | G | 148 | A | 1 |
| 6.57 | B | 64 | Q | 65 | M | 2 |
| 6.57 | A | 148 | L | 149 | L | 2 |
| 6.77 | A | 350 | I | 352 | I | 2 |
| 6.77 | A | 247 | S | 249 | S | 2 |
| 7.01 | A | 157 | M | 158 | M | 2 |
| 7.31 | A | 380 | G | 382 | G | 2 |
| 7.42 | A | 322 | Q | 324 | Q | 2 |
| 7.47 | A | 377 | L | 379 | M | 3 |
| 8.11 | A | 353 | S | 355 | T | 4 |
| 8.24 | A | 88 | L | 89 | L | 4 |
| 8.33 | A | 289 | G | 291 | G | 4 |
| 8.35 | A | 379 | I | 381 | I | 4 |
| 8.57 | A | 160 | T | 161 | T | 4 |
| 8.85 | A | 161 | A | 162 | A | 4 |
| 8.94 | A | 292 | P | 294 | P | 4 |
| 8.94 | A | 248 | G | 250 | G | 4 |
| 9.03 | A | 343 | A | 345 | G | 5 |
| 9.26 | A | 119 | N4 | 120 | N4 | 5 |
| 9.48 | A | 57 | V | 57 | V | 5 |
| 9.66 | A | 156 | H | 157 | H | 5 |
| 9.85 | A | 246 | A | 248 | A | 5 |
| 10.06 | A | 249 | L | 251 | L | 5 |
| 10.33 | A | 347 | G | 349 | G | 5 |
| 10.41 | A | 158 | G | 159 | G | 5 |
| 10.41 | A | 383 | N4 | 385 | Q | 6 |
| 10.44 | A | 91 | S | 92 | S | 6 |
| 10.56 | A | 349 | P | 351 | P | 6 |
| 10.61 | A | 164 | V | 165 | V | 6 |
| 10.65 | A | 381 | G | 383 | G | 6 |
| 10.66 | A | 90 | G | 91 | G | 6 |
| 10.66 | A | 283 | V | 285 | V | 6 |
| 10.75 | A | 235 | F | 236 | F | 6 |
| 10.90 | A | 382 | G | 384 | G | 6 |
| 11.00 | A | 326 | V | 328 | V | 6 |
| 11.01 | A | 323 | A | 325 | A | 6 |
| 11.08 | A | 357 | I | 359 | I | 6 |
| 11.29 | A | 351 | G | 353 | G | 6 |
| 11.34 | A | 291 | G | 293 | G | 6 |
| 11.49 | A | 384 | G | 386 | G | 6 |
| 11.56 | A | 321 | A | 323 | A | 6 |
| 11.58 | B | 65 | N | 66 | Y | 7 |
| 11.81 | A | 344 | I | 346 | I | 7 |
| 11.88 | A | 150 | D | 151 | D | 7 |
| 11.93 | A | 290 | T | 292 | I | 8 |
| 11.96 | A | 87 | Q | 88 | R | 9 |
| 12.07 | A | 315 | A | 317 | A | 9 |
| 12.10 | A | 356 | R | 358 | L | 10 |
| 12.14 | A | 376 | T | 378 | T | 10 |
| 12.25 | A | 58 | L | 58 | I | 11 |
| 12.28 | A | 86 | N | 87 | N | 11 |
| 12.41 | A | 241 | V | 243 | V | 11 |
| 12.55 | A | 144 | I | 146 | L | 12 |
| 12.60 | A | 149 | T | 150 | H | 13 |
| 12.62 | A | 385 | V | 387 | I | 14 |

Differences between PhbA and BktB active sites are ordered by distance from PhbA priming acetyl-CoA acetyl carbonyl carbon. Note that overall the two thiolases share only 52% sequence identity, however their active sites are highly conserved, with only 5 amino acid differences within a 10 Å shell from the catalytic Cys89/90 residues. Table II was generated by aligning Chains A and B of the PhbA structure (PDB: 1M3Z) to Chains A and B of the BktB crystal structure (PDB: 4NZS) using the super command in Pymol. Residues in bold indicate catalytic residues (C89/C90, H348/H350, C378/C380). Residues italicized indicate residues mutated in both BktB and PhbA (M157/M158, M288/M290). Residues with a single underline indicate those mutated only in PhbA (L377, L88). Residues with a double underline indicate those mutated in BktB only (Y66).

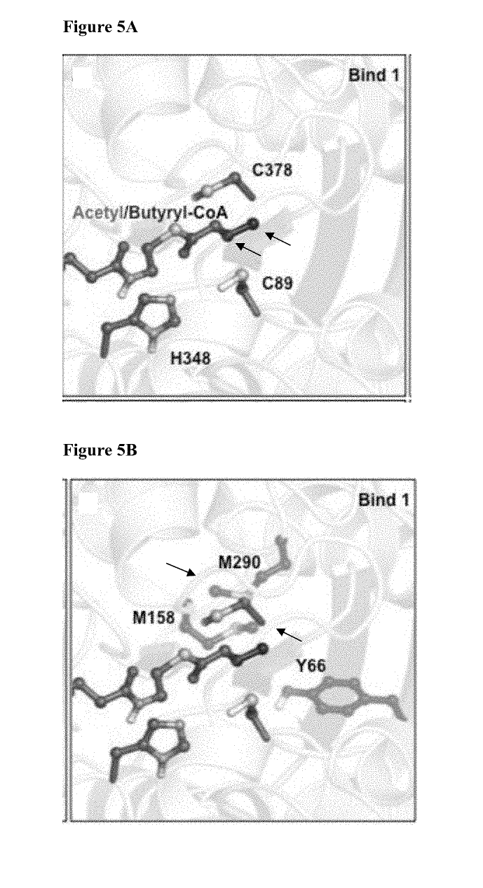

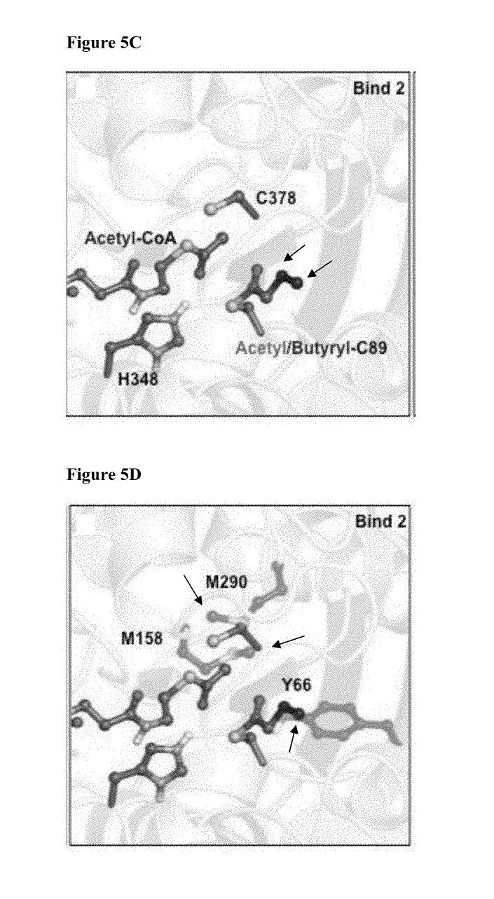

The results of the computational model applied to the BktB thiolase are shown in Table III. Given the active-site similarity, it was not surprising that two BktB residues with analogous PhbA positions (M157/M158, M288/M290) were also predicted to improve BA/AA selectivity. Additionally, a position unique to BktB was predicted, Y66, which is part of a loop that comprises the major structural difference between the PhbA and BktB active sites. The positions of the BktB residues chosen for mutagenesis relative to the Bind 1 and Bind 2 conformations are shown in FIGS. 5A-5D.

TABLE III
Energetic calculations of C. necator BktB mutants selected for experimental testing

| | Bind 1 | | | Bind 2 | | |
|---|---|---|---|---|---|---|
| Mutant | ΔΔE_{Bind B}^{Mut-WT} | ΔΔE_{Bind A}^{Mut-WT} | ΔΔΔE_{Bind B-A}^{Mut-WT} | ΔΔE_{Bind B}^{Mut-WT} | ΔΔE_{Bind A}^{Mut-WT} | ΔΔΔE_{Bind B-A}^{Mut-WT} |
| M158S | −2.18 | 1.27* | −3.45 | −1.33 | 1.03* | −2.36 |
| M158A | −1.86 | 1.58* | −3.44 | −1.54 | 1.49* | −3.03 |
| M158G | −1.54 | 1.61* | −3.15 | −0.16 | 1.99* | −2.15 |
| M290S | −21.81 | 0.45* | −22.26 | 0.13* | 0.31* | −0.18 |
| M290A | −22.03 | 0.55* | −22.59 | 0.18* | 0.42* | −0.23 |
| M290G | −21.52 | 0.64* | −22.16 | 0.29* | 0.51* | −0.22 |
| Y66Q | −0.36 | 0.12* | −0.48 | −2.52 | 0.08* | −2.60 |
| Y66N | 0.10* | 0.09* | 0.01* | −2.49 | 0.12* | −2.61 |
| Y66V | 0.26* | 0.12* | 0.14* | −2.49 | 0.11* | −2.60 |
| Y66T | 0.09* | 0.12* | −0.04 | −2.46 | 0.12* | −2.58 |
| Y66S | −0.32 | 0.11* | −0.43 | −2.48 | 0.13* | −2.61 |
| Y66A | 0.08* | 0.12* | −0.04 | −2.44 | 0.14* | −2.58 |
| Y66G | 0.18* | 0.12* | 0.05* | −2.45 | 0.15* | −2.60 |

In the Bind 1 column, ΔΔE_{Bind B}^{Mut-WT} is the difference in binding energies between mutant and wild type bound to butyryl-CoA with free C90, ΔΔE_{Bind A}^{Mut-WT} is the difference in binding energies between mutant and wild type bound to acetyl-CoA with C90 unacylated, and ΔΔΔE_{Bind B-A}^{Mut-WT} is the difference between ΔΔE_{Bind B}^{Mut-WT} and ΔΔE_{Bind A}^{Mut-WT} corresponding to the differential specificities for mutant versus wild type for binding butyryl-CoA versus acetyl-CoA as the priming acyl-CoA.

In the Bind 2 column ΔΔE_{Bind B}^{Mut-WT} is the difference in binding energies between mutant and wild type bound to acetyl-CoA with butyrylated C90, ΔΔE_{Bind A}^{Mut-WT} is the difference in binding energies between mutant and wild type bound to acetyl-CoA with acetylated C90, and ΔΔΔE_{Bind B-A}^{Mut-WT} is the difference between ΔΔE_{Bind B}^{Mut-WT} and ΔΔE_{Bind A}^{Mut-WT} corresponding to the differential specificities for mutant versus wild type for binding acetyl-coA as the extending CoA with C90 either butyrylated or acetylated. Negative energies are indicated with a “*”, and positive energies are unmarked. All energies are reported in kcal/mol.

### BktB Thiolases Enable Synthesis of PHAs Enriched in 3HHx

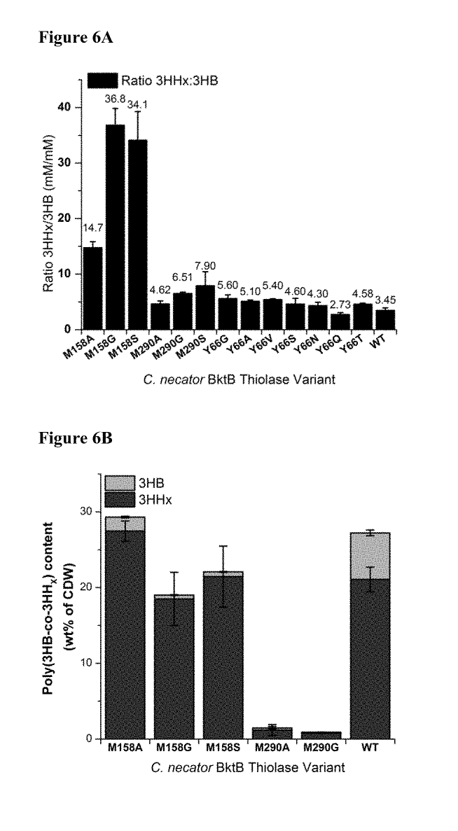

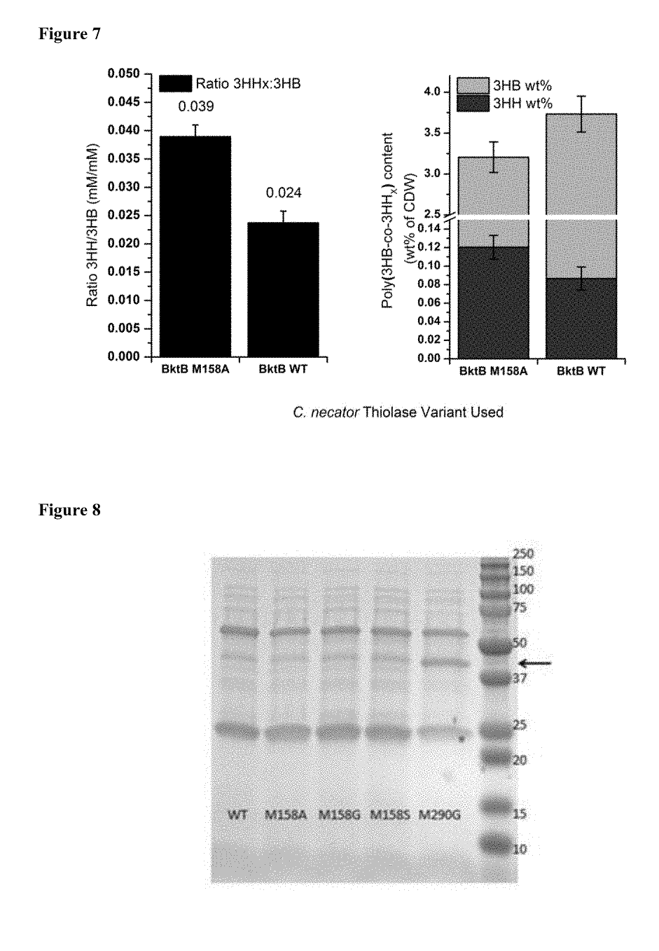

The BktB thiolase has been previously used to achieve synthesis of longer (>C4) and branched chain acids, aldehydes and alcohols by the same CoA dependent pathway (Cheong et al., 2016; Dhamankar et al., 2014; Kim et al., 2015). Of the M158, M290 and Y66 mutants assayed, the M158 mutants resulted in the highest selectivity ratios, with M158G and M158S exhibiting selectivity ratios 10-fold greater than wild type for 3HHx in PHAs (FIG. 6A). Based on previous reports of their activities, it was not surprising that wild type baseline selectivity was higher for BktB at 3.45 compared to PhbA at 0.292 (Slater et al., 1998). The PHA polymers isolated from E. coli strains expressing these mutants varied from 77 to 97 mol % 3HHx (Table IV), with BktB M158A mutant resulting in the highest yields of 3HHx as a percentage of the CDW (FIG. 6B). Protein gels of lysates of strains expressing wild type vs. mutant enzymes showed no significant difference in the soluble expression level of the thiolase enzymes, pointing to differences in the activities of these enzymes (FIG. 8). It was surprising that the M290 mutants resulted in very low yields of PHAs in vivo. Although it is possible that BktB expression or solubility was affected as a result of this mutation, soluble expression was detected via a protein gel.

TABLE IV
Composition of PHAs extracted from engineered E. coli strains overexpressing
different thiolases and grown on glucose with fed butyrate.

| Thiolase | CDW (g/L) | PHA Content (wt %) | Mol % 3HHx |
|---|---|---|---|
| Z. ramigera PhbA WT | 0.52 ± 0.022 | 4.3 ± 0.41 | 22.6 ± 1.81 |
| Z. ramigera PhbA M158A | 1.26 ± 0.073 | 0.41 ± 0.046 | 83.9 ± 2.7 |
| Z. ramigera PhbA M158G | 0.72 ± 0.34 | 0.083 ± 0.08 | 85.6 ± 2.15 |
| Z. ramigera PhbA M158S | 0.96 ± 0.42 | 0.51 ± 0.17 | 84.2 ± 4.02 |
| C. necator BktB WT | 1.04 ± 0.13 | 27.2 ± 1.2 | 77.3 ± 2.3 |
| C. necator BktB M158A | 0.81 ± 0.16 | 29.3 ± 1.4 | 93.6 ± 0.42 |
| C. necator BktB M158G | 0.71 ± 0.17 | 18.9 ± 3.55 | 97.3 ± 0.43 |
| C. necator BktB M158S | 0.88 ± 0.16 | 22.1 ± 4.08 | 97.3 ± 0.22 |
| C. necator BktB M290A | 0.65 ± 0.23 | 1.47 ± 0.90 | 81.7 ± 1.4 |
| C. necator BktB M290G | 0.54 ± 0.04 | 0.90 ± 0.05 | 86.7 ± 0.46 |

In Vitro Characterization of BktB Thiolase Mutants with Highest Selectivity Ratios

Having achieved increased selectivity ratios of the 3HHx:3HB in the PHA polymers with the computationally designed mutants, the effects of the M158 mutations on thiolase activity were studied next. The in vivo data suggested that increased selectivity ratios were obtained due to decreased activity of these mutant thiolases for the formation of the AA condensation product (and subsequently 3HB) and not due to increased activity towards the condensation of butyryl-CoA with acetyl-CoA, which results in the formation of 3-oxo-hexanoyl-CoA (and 3HH-CoA upon reduction). Thus, it was sought to purify and assay both the mutant and wild type BktB thiolases in vitro to remove many of the confounding variables present in vivo. For example, differences in stability of the enzymes as well as fluctuating pools of substrates and coenzymes could influence thiolase activity. Further, activities of the downstream enzymes could also influence the final product distribution. Each wild type and mutant enzyme was purified as a His-tag fusion to homogeneity and assayed in the condensation direction with acetyl-CoA, and thiolysis directions with acetoacetyl-CoA (AA) and 3-oxo-hexanoyl-CoA (BA). Activity measurements of the BktB M290A mutant revealed a very low k_{cat} for the condensation of two acetyl-CoAs consistent with in vivo observations. In vitro characterization of the wild type BktB and M158A BktB enzymes revealed a 10-fold lower catalytic activity of the mutant towards the condensation of two acetyl-CoA molecules (Table V). This result was consistent with in vivo observations of reduced 3HB product formation which arises from the condensation of two acetyl-CoAs. From the in vitro kinetic parameters it can be concluded that the M158 mutants do indeed have lower catalytic efficiencies towards the formation and degradation of AA, the C4 product (1.52×104 vs. 1.46×103 M−1 sec−1, WT vs mutant); whereas the thiolysis k_{cat}/Kₘ value towards the degradation of BA, the C6 product, is 3-fold higher as compared to wild type (2.67×105 vs. 9.82×105, WT vs mutant, Table V). In all, there was an 80-fold improvement in the selectivity ratio of the M158A thiolase as compared to wild type BktB.

TABLE V
In vitro kinetic characterization of thiolase variants

| | k_{cat} | Kₘ | k_{cat}/Kₘ | C6/C4 |
|---|---|---|---|---|
| Reaction | (sec⁻¹) | (μM) | (M⁻¹sec⁻¹) | Selectivity |
| BktB WT C4 Condensation | 14.1 | 919 | 1.52 × 10⁴ | N/A |
| BktB M158A C4 Condensation | 1.33 | 913 | 1.46 × 10³ | |
| BktB WT C4 Thiolysis | 148 | 17.5 | 8.45 × 10⁶ | 0.032 |
| BktB WT C6 Thiolysis | 4.06 | 15.2 | 2.67 × 10⁵ | |
| BktB M158A C4 Thiolysis | 4.63 | 14.1 | 3.28 × 10⁵ | 2.99 |
| BktB M158A C6 Thiolysis | 16.7 | 17 | 9.82 × 10⁵ | |

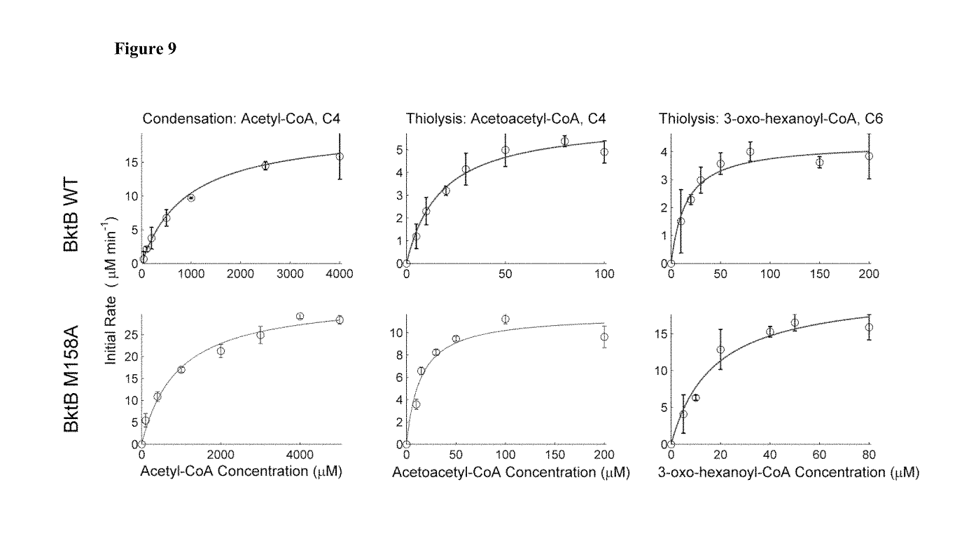

In vitro characterization of C. necator wild type BktB and BktB variant thiolases in the forward direction (condensation) with C4, and reverse direction (thiolysis) with both C4 and C6 substrates. Catalytic parameters were computed from fits to the Michaelis Menten equation (FIG. 9).

Using C. necator BktB M158A Mutant Allows for Biosynthesis of PHAs Enriched in 3HHx from Glucose as Sole Carbon Source

Having demonstrated increased selectivity for the BktB M158A variant in vivo while supplying both butyrate and glucose, it was next determined whether this mutant could allow for more selective synthesis of longer chain product using glucose as the sole source of carbon. The same model system as was used as previously, except that additional enzymes that would allow for conversion of 3-hydroxybutyryl-CoA to butyryl-CoA were overexpressed. Trans-enoyl-CoA reductase, Ter from Treponema denticola was cloned into the first MCS of pCDFDue t and enoyl-CoA hydratase, PhaJ4b from C. necator was cloned into an operon with PhaC2 generating pCDFDuet(terTd)-(phaC2-phaJ4). This vector, along with pETDuet(BktB WT or M158A)-(phaB), was used to transform E. coli MG1655(DE3), and the strain was grown in M9 medium with glucose as a sole carbon source. FIG. 7 shows that the residual activity of BktB M158A towards the condensation of two acetyl-CoAs was sufficient to allow for formation of butyryl-CoA and subsequently 3-hydroxybutyryl-CoA. Using the BktB M158A variant led to an almost 2-fold increase in selectivity for the 3HHx monomer as compared to using wild type BktB, though the overall yield of PHAs was low. A nearly wild type E. coli was used for all production experiments in this work. It is likely that additional strain engineering to increase precursor supply and eliminate competing pathways may lead to increased product yields.

### Materials and Methods

### Chemicals and Reagents:

All chemicals were obtained from Sigma Aldrich unless stated otherwise. Protein purification reagents were purchased from BioRad Laboratories (Hercules, Calif.).

### Strain and Plasmid Construction:

Escherichia coli MG1655 K12 (DE3) was used as the host for all production experiments. pCDFDuet-pct-phaC2 was constructed by restriction enzyme cloning. First, pct from M. elsdenii was amplified using Q5 Polymerase (New England Biolabs, Ipswish, Mass.) from M. elsdenii gDNA. PhaC2 was synthesized as a codon optimized gBlock from ThermoFischer and digested with the respective restriction enzymes. Construction of pETDuet-bktB-phaB is described in Martin et al. (2013). This plasmid served as the template for generating BktB mutants. Primer sequences can be found in Table VI.

TABLE VI
Primers and Strains Used in this Study

| | | SEQ |
|---|---|---|
| | | ID |
| Primer Name | Sequence | NO: |
| bktB_m158a_F | ggcggtcacgcccgcgtggatgcgatgg | 5 |
| bktB_m158a_R | ccatcgcatccacgcgggcgtgaccgcc | 6 |
| bktB_m158g_F | ccatcgcatccacgggggcgtgaccgcc | 7 |
| bktB_m158g_R | ggcggtcacgcccccgtggatgcgatgg | 8 |
| bktB_m158s_F | cccttccatcgcatccacagcggcgtgaccg | 9 |
| bktB_m158s_R | cggtcacgccgctgtggatgcgatggaaggg | 10 |
| bktB_m290a_F | ggacccgaaggcggcgggcatcggcccg | 11 |
| bktB_m290a_R | cgggccgatgcccgccgccttcgggtcc | 12 |
| bktB_m290g_F | ggacccgaaggcggggggcatcggcccg | 13 |
| bktB_m290g_R | cgggccgatgccccccgccttcgggtcc | 14 |
| bktB_m290s_F | tggacccgaaggcgagcggcatcggccc | 15 |
| bktB_m290s_R | gggccgatgccgctcgccttcgggtcca | 16 |
| bktB_y66a_F | gccgcgcgacatggctctgggccgcgtc | 17 |
| bktB_y66a_R | gacgcggcccagagccatgtcgcgcggc | 18 |
| bktB_y66g_F | gccgcgcgacatgggtctgggccgcgtc | 19 |
| bktB_y66g_R | gacgcggcccagacccatgtcgcgcggc | 20 |
| bktB_y66n_F | ccgcgcgacatgaatctgggccgcg | 21 |
| bktB_y66n_R | cgcggcccagattcatgtcgcgcgg | 22 |
| bktB_y66q_F | gccgcgcgacatgcagctgggccgcgtcg | 23 |
| bktB_y66q_R | cgacgcggcccagctgcatgtcgcgcggc | 24 |
| bktB_y66s_F | gccgcgcgacatgagtctgggccgcgtc | 25 |
| bktB_y66s_R | gacgcggcccagactcatgtcgcgcggc | 26 |
| bktB_y66t_F | gccgcgcgacatgactctgggccgcgtc | 27 |
| bktB_y66t_R | gacgcggcccagagtcatgtcgcgcggc | 28 |
| bktB_y66v_F | gccgcgcgacatggttctgggccgcgtc | 29 |
| bktB_y66v_R | gacgcggcccagaaccatgtcgcgcggc | 30 |
| BktB_SeqF | gcagcctgaaggatgtg | 31 |
| BktB_SeqR | Gcgccttcaccgtgatc | 32 |
| PhaC2_F | gctagcgaattcctacaaggagaacaaac | 33 |
| PhaC2_R | ctagaggatccccgggctcgag | 34 |
| phaB_F | aacctgtattttcagggcatgactcagcgcatt | 35 |
| phab_R | gctcgagaattccatggctcagcccatatgcag | 36 |
| phbA_L378A_F | gggtctcgccacggcctgcatcggcggc | 37 |
| phbA_L378A_R | gccgccgatgcaggccgtggcgagaccc | 38 |
| phbA_L378G_F | gggtctcgccacgggctgcatcggcggc | 39 |
| phbA_L378G_R | gccgccgatgcagcccgtggcgagaccc | 40 |
| phbA_L378S_F | gggtctcgccacgagctgcatcggcggc | 41 |
| phbA_L378S_R | gccgccgatgcagctcgtggcgagaccc | 42 |
| phbA_L89A_F | gcccgagccgcaagcctggttcatgccc | 43 |
| phbA_L89A_R | gggcatgaaccaggcttgcggctcgggc | 44 |
| phbA_L89G_F | gcccgagccgcaaccctggttcatgccc | 45 |
| phbA_L89G_R | gggcatgaaccagggttgcggctcgggc | 46 |
| phbA_L89S_F | ggcccgagccgcaactctggttcatgcccc | 47 |
| phbA_L89S_R | ggggcatgaaccagagttgcggctcgggcc | 48 |
| phbA_M158A_F | ctacggctaccacgcgggcacgaccgcc | 49 |
| phbA_M158A_R | ggcggtcgtgcccgcgtggtagccgtag | 50 |
| phbA_M158G_F | ctacggctaccacgggggcacgaccgcc | 51 |
| phbA_M158G_R | ggcggtcgtgcccccgtggtagccgtag | 52 |
| phbA_M158S_F | gccttctacggctaccacagcggcacgaccg | 53 |
| phbA_M158S_R | cggtcgtgccgctgtggtagccgtagaaggc | 54 |
| phbA_M289A_F | cgatcccaaggtcgcgggcaccggcccg | 55 |
| phbA_M289A_R | cgggccggtgcccgcgaccttgggatcg | 56 |
| phbA_M289G_F | cgatcccaaggtcgggggcaccggcccg | 57 |
| phbA_M289G_R | cgggccggtgcccccgaccttgggatcg | 58 |
| phbA_M289S_F | cgtcgatcccaaggtcagcggcaccggc | 59 |
| phbA_M289S_R | gccggtgccgctgaccttgggatcgacg | 60 |

Codon Optimized Sequence of PhaC2 from R. aetherivorans I24

This sequence was cloned into the second MCS of pCDF_pct_{M. elsdenii} using the NdeI and XhoI restriction sites, which are underlined in the sequence below.

(SEQ ID NO: 61)
GATATACATATGGCACAGGCACGTACCGTTATTGGTGAAAGCGTTGAAGA
AAGCATTGGTGGTGGTGAAGATGTTGCACCGCCTCGTCTGGGTCCGGCAG
TTGGTGCACTGGCAGATGTTTTTGGTCATGGTCGTGCAGTTGCACGTCAT
GGTGTTAGCTTTGGTCGTGAACTGGCAAAAATTGCAGTTGGTCGTAGCAC
CGTTGCACCGGCAAAAGGTGATCGTCGTTTTGCAGATAGCGCATGGTCAG
CAAATCCGGCATATCGTCGCCTGGGTCAGACCTATCTGGCAGCAACCGAA
GCAGTTGATGGTGTTGTTGATGAAGTGGGTCGTGCAATTGGTCCGCGTCG
TACCGCAGAAGCACGTTTTGCCGCAGATATTCTGACCGCAGCACTGGCAC
CGACCAATTATCTGTGGACCAATCCGGCAGCACTGAAAGAAGCATTTGAT
ACCGCAGGTCTGAGCCTGGCACGTGGCACCAAACATTTTGTTAGCGATCT
GATTGAAAATCGTGGTATGCCGAGCATGGTTCAGCGTGGTGCATTTACCG
TTGGTAAAGATCTGGCAGTTACACCGGGTGCAGTTATTAGCCGTGATGAA
GTTGCCGAAGTTCTGCAGTATACCCCGACCACCGAAACCGTTCGTCGTCG
TCCGGTTCTGGTTGTTCCGCCTCCGATTGGTCGTTATTACTTTCTGGATC
TGCGTCCGGGTCGTAGCTTTGTTGAATATAGTGTTGGTCGTGGCCTGCAG
ACCTTTCTGCTGAGCTGGCGTAATCCGACCGCAGAACAGGGTGATTGGGA
TTTTGATACCTATGCAGGTCGTGTTATTCGTGCAATCGATGAAGTTCGTG
AAATCACCGGTAGTGATGATGTTAATCTGATTGGTTTTTGTGCCGGTGGT
ATTATTGCAACCACCGTTCTGAATCACCTGGCAGCCCAGGGTGATACCCG
TGTTCATAGCATGGCCTATGCAGTTACCATGCTGGATTTTGGTGATCCGG
CACTGCTGGGTGCATTTGCCCGTCCTGGTCTGATTCGTTTTGCCAAAGGT
CGTAGCCGTCGTAAAGGTATTATTAGCGCACGTGATATGGGTAGCGCATT
TACCTGGATGCGTCCGAATGATCTGGTTTTTAACTATGTGGTGAACAACT
ATCTGATGGGTCGTACCCCTCCTGCCTTTGATATTCTGGCATGGAATGAT
GATGGTACAAATCTGCCTGGTGCCCTGCATGGCCAGTTTCTGGATATTTT
TCGTGATAATGTTCTGGTGGAACCGGGTCGTCTGGCCGTTCTGGGTACAC
CGGTTGATCTGAAAAGCATTACCGTTCCGACCTTTGTGAGCGGTGCAATT
GCCGATCATCTGACCGCGTGGCGTAATTGTTATCGTACCACACAGCTGCT
GGGAGGTGAAACCGAATTTGCACTGAGCTTTAGCGGTCATATTGCAAGCC
TGGTTAATCCTCCGGGTAATCCGAAAGCACATTATTGGACCGGTGGCACA
CCGGGTCCGGATCCTGATGCATGGCTGGAAAATGCAGAACGTCAGCAGGG
TAGTTGGTGGCAGGCCTGGTCAGATTGGGTTCTGGCACGCGGTGGCGAAG
AAACAGCAGCACCGGATGCACCGGGTAGTGCACAGCATCCTGCACTGGAT
GCCGCACCGGGTCGCTATGTTCGTGATCTGCCTGCAGGTTAACTCGAGTC
TGGT

### Culture Conditions and Strain Propagation

E. coli DH5a was used for construction and maintenance of all plasmids. For PHA production experiments, E. coli MG1655 K12 (DE3) was transformed by electroporation with pCDFDuet-pct-phaC2 and a pETDuet plasmid with a given thiolase variant and phaB. For every production experiment, three individual colonies were picked and grown overnight in LB medium containing carbenicilin (50 μg/mL) and streptomycin (50 μg/mL) at 30° C., 250 rpm. A 250-mL shake flask containing 50 mL of M9 minimal medium with 15 g/L glucose was used for production experiments and inoculated with 1% v/v of the overnight starter culture.

Expression of heterologous genes was induced by addition of IPTG to 100 μM final concentration when OD₆₀₀ was 0.7-1.0. Butyrate was added to 15 mM final concentrations from a neutralized sterile stock solution at induction. Cells were harvested by centrifugation and washed twice with water before freezing at −80° C. and lyophilization for polymer extraction and derivatization. For analysis of free acids, cell-free culture supernatants were analyzed directly by HPLC.

### Site Specific Mutagenesis

All point mutants were made using the QuikChange Lightning XL Kit from Agilent Technologies according to the manufacturer's protocols (Agilent Technologies, Santa Clara, Calif.), except that DH5α cells were used for transformation of QuikChange products. The online primer design tool (www.genomics.agilent.com/primerDesignProgram.jsp) was used to generate the mutagenesis primers to be used in the thermal cycling reaction. Primer sequences can be found in the Table VI. Products of this reaction were used to transform chemically competent E. coli DH5α and plated on selective medium after recovery in SOC. Individual colonies were selected and mutations confirmed by sequencing (GeneWiz, Cambridge, Mass.).

### Product Analysis

Acidic methanolysis was performed as described in Brandl et al. (1988) to analyze PHA composition and is briefly described below. Cells were harvested by centrifugation and washed twice with water. The cells were then frozen at −80° C. Lyophilized cells were weighed to determine the cell dry weight (CDW). Then, 5-20 mg of dried cells was used for methanolysis to determine PHA polymer composition by GC/MS. Hexanoic acid was added as an internal standard to a final concentration of 2.5 mM. In short, 1 mL chloroform, 0.85 mL methanol and 0.15 mL concentrated H₂SO₄ was added to each sample in a screw-capped tube with threads wrapped with PTFE tape. The samples were then boiled for 1.5-2 hours at 100° C. on a heating block with intermittent manual mixing. After boiling, the tubes were cooled and placed on ice, followed by addition of 0.5 mL water and vortexing for 1 minute. Tubes were centrifuged to achieve phase separation. The bottom chloroform layer was then transferred into a glass vial, dried over MgSO₄, and filtered through a 0.45 μm PTFE filter into a GC vial.

Derivatized 3HAs were analyzed on an Agilent 7890B/5977A GC/MS with a VF-WAX column (30 m×250 μm×0.5 μm). The following method parameters were used: inlet temperature of 220° C., initial oven temperature of 80° C. and a linear ramp rate of 10° C./min until final oven temperature of 220° C., with a 10:1 split ratio. An FID detector was used for quantification of methyl-3HB and methyl-3HH. Quantification of free acids, 3-hydroxyhexanoic and 3-hydroxybutyric acids, was performed by HPLC. One mL of culture was harvested at induction and at 72 hours post induction and centrifuged at maximum speed for 6 minutes. A sample of the supernatant was then run on an Aminex HP-87x (BioRad, Hercules, Calif.) column on an Agilent 1200 HPLC instrument equipped with an RID detector. 5 mM sulfuric acid was used as the mobile phase at 0.6 mL/min with column temperature held at 35° C.

### Protein Purification

Thiolase variants were subcloned into a protein expression vector pTev5 with an N-terminal hexa-histidine tag using CPEC cloning with primers listed Table VI. E. coli BL21(DE3) was used as the host for protein expression. One liter of culture was grown in TB medium with glycerol at 30° C. and induced with 100 μM IPTG when OD₆₀₀ was ˜0.5. Cells were harvested 15-18 hours post-induction by centrifugation and resuspended in 2.5× vol/wt buffer containing 50 mM Tris-HCl pH 8.0, 500 mM NaCl and 10% vol/vol glycerol. Lysozyme was added to 1 mg/mL final concentration and cells were lysed by sonication. Protein purification was then performed as described previously (McMahon and Prather, 2014). After purification, proteins were exchanged into storage buffer (50 mM Tris pH 8.0, 50 mM NaCl and 10% vol/vol glycerol), flash frozen in small aliquots and stored at −80° C. Protein concentration was determined by a Bradford assay using BSA as standard. PhaB reductase from C. necator, which was used as a coupling enzyme in condensation assays, was purified in the same manner as described above.

### Enzyme Assays

Thiolase variants were assayed in both condensation and thiolysis directions. The condensation assay was performed akin to that described previously (Bond-Watts et al., 2011), except at pH 7.0 and coupled to PhaB reductase (from C. necator). Each reaction contained 100 mM Tris pH 7 buffer, 100 μg/mL NADPH, and varying amounts of acetyl-CoA, and reaction progress was monitored by a decrease in A₃₄₀ nm corresponding to NADPH consumption on a Beckman-Coulter DU800 spectrophotometer.

Thiolases were also assayed in the thermodynamically favored thiolysis direction with acetoacetyl-CoA and 3-oxo-hexanoyl-CoA. Each assay contained 100 mM Tris pH 7.0, 10 mM MgCl₂, 200 μM CoASH, an appropriate amount of enzyme, and varying substrate concentration. A decrease in A₃₀₃, corresponding to consumption of the Mg-keto-acyl-CoA complex was measured spectrophotometrically. The extinction coefficient for acetoacetyl-CoA was determined to be 4.22 μM⁻¹ cm⁻¹ under the enzymatic conditions. Concentrations of all enzymes used in the assays were such that the reaction rate was linear for at least 0.5 minutes. Enzymes were diluted in pH 7 dilution buffer (100 mM Tris pH 7, 50 mM NaCl and 10% vol/vol glycerol). Each substrate concentration was assayed at least in duplicate. Generated concentration vs. initial rate curves were fit to the Michaelis-Menten equation, from which catalytic parameters (k_{cat} and Kₘ) were determined using the nlinfit routine in MATLAB.

Synthesis of 3-oxo-hexanoyl-CoA

The generalized synthesis is outlined in Scheme I below and was inspired from the synthesis of ethylmalonyl-CoA by Dunn et al. (2014) and adapted by M. Blaissey and M. C. Y. Chang. 3-oxo-hexanoic acid methyl ester was purchased from Alfa Aesar. 1 mmol of the ester was allowed to react with 1.2 mmol aqueous NaOH at room temperature overnight. The reaction was then neutralized to pH 7.0 and extracted three times with ethyl acetate, dried over MgSO₄ and solvent evaporated. 3-oxo-hexanoic acid appeared as a white solid. This crude solid was used in subsequent thioesterification with 1.2 mmol of thiophenol, 1.5 mmol diisopropylcarbodiimide and 2 mg of dimethylaminopyridine in 10 mL of ethyl acetate. The reaction was carried out on ice for 2 hours, followed by 2 hours at room temperature, after which the white precipitate was filtered off and the filtrate extracted with saturated sodium bicarbonate. The organic layer was then dried over MgSO₄ and solvent evaporated. Crude thiophenol-coupled product was then re-dissolved in 200 μL acetonitrile and added to 1 mL of 0.5 M NaHCO₃ on an ice bath. 25 mg of CoASH was added and the reaction allowed to proceed for 1 hour on ice and then 1 hour at room temperature. The reaction was quenched with 50% formic acid, and extracted with diethyl ether. Final 3-oxo-hexanoyl-CoA product was purified by HPLC with 25 mM ammonium acetate pH 4.5 and 20% acetonitrile in water as the movie phases using a linear gradient from 1% v/v acetonitrile to 20% over 25 minutes on an Agilent Zorbax Eclipse XDB C18 column. Identity of the compound was verified by mass spectrometry. Finally, the purified product was desalted on the same column but with only water and acetonitrile as movie phases.

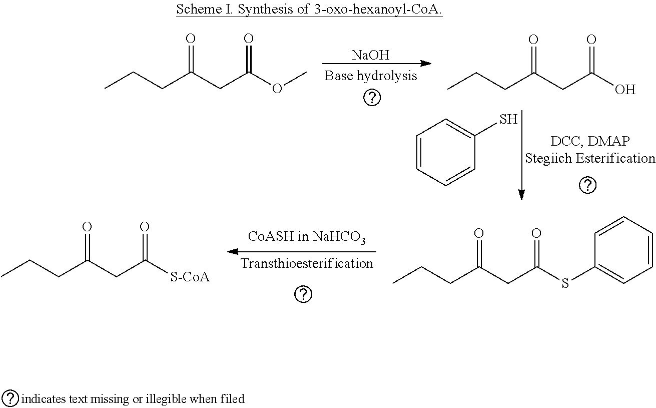

### Starting X-Ray Structures

For PhbA calculations with butyryl-CoA and acetyl-CoA bound with C89 unacylated (“Bind 1”), 1M3Z (C89A mutant with acetyl-CoA bound) was used as the starting crystal structure with the C89 built into the structure using the same dihedral angles as the C89 of the unliganded wild type PhbA thiolase structure 1DLU (Kursula et al., 2002; Modis and Wierenga, 2000). For PhbA calculations with C89 acylated (“Bind 2”), 1DM3 (wild type enzyme with acetyl-CoA bound and C89 acetylated) was used as the starting structure (Modis and Wierenga, 2000). For BktB calculations, 4NZS (wild type enzyme, unliganded) was used as the starting crystal structure (Kim et al., 2014). All crystal structures were prepared for computer modeling with the CHARMM36 force field (Brooks et al., 2009) using the methodology outlined in Lippow et al. (2007). CHARMM parameters for acetyl-CoA were taken from Aleksandrov and Field (2011).

### Computational Methodology

Mathematically, calculations for each of the two binding events sought to optimize:

ΔΔΔE_{Bind B-Bind A}^{Mut-WT}=ΔΔE_{Bind B}^{Mut-WT}−ΔΔE_{Bind A}^{Mut-WT}

where the subscript Bind B refers to the structure with a butyryl group in either the first or second binding event and the subscript Bind A refers to the corresponding structure with an acetyl group in either Bind 1 or Bind 2. Bind B refers to the bound conformation leading to BA production in either step and Bind A refers to the bound conformation leading to AA production in either step. For example, the structure optimized for Bind B in the first binding event corresponds to step 2 of FIG. 1C where R is a butyryl group, and the structure optimized for Bind A in the first binding event corresponds to step 2 of FIG. 1C where R is an acetyl group.

Thus the optimization sought to minimize the difference of the following two terms:

ΔΔE_{Bind B}^{Mut-WT}=ΔΔE_{Bind B}^{WT}−ΔΔE_{Bind B}^{WT}

ΔΔE_{Bind A}^{Mut-WT}=ΔΔE_{Bind A}^{WT}−ΔΔE_{Bind A}^{WT}

where,

ΔE_{Bind B}^{Mut}=ΔE_{Complex B}^{Mut}−ΔE_{Receptor}^{Mut}−ΔE_{Ligand B}^{Mut}

ΔE_{Bind B}^{WT}=ΔE_{Complex B}^{WT}−ΔE_{Receptor}^{WT}−ΔE_{Ligand B}^{WT}

ΔE_{Bind A}^{Mut}=ΔE_{Complex A}^{Mut}−ΔE_{Receptor}^{Mut}−ΔE_{Ligand A}^{Mut}

ΔE_{Bind A}^{WT}=ΔE_{Complex A}^{WT}−ΔE_{Receptor}^{WT}−ΔE_{Ligand A}^{WT}

subject to

ΔΔE_{Fold B}^{Mut-WT}<Fold Cutoff

ΔΔE_{Fold A}^{Mut-WT}<Fold Cutoff

where

ΔΔE_{Fold B}^{Mut-WT}=ΔE_{Fold B}^{Mut}−ΔE_{Fold B}^{WT}

ΔΔE_{Fold A}^{Mut-WT}=ΔE_{Fold A}^{Mut}−ΔE_{Fold A}^{WT}

and

ΔE_{Fold B}^{Mut}=ΔE_{Receptor B}^{Mut}−ΔE_{Unfolded}^{Mut}

ΔE_{Fold B}^{WT}=ΔE_{Receptor B}^{WT}−ΔE_{Unfolded}^{WT}

ΔE_{Fold A}^{Mut}=ΔE_{Receptor A}^{Mut}−ΔE_{Unfolded}^{Mut}

ΔE_{Fold B}^{WT}=ΔE_{Receptor B}^{WT}−ΔE_{Unfolded}^{WT}

To accomplish this, for each mutant and for both binding events, four separate global minimum energy conformations (GMEC) were computed using the Dead End Elimination/A* based approach as implemented by Lippow et al. (2007).

ΔE_{Fold+Bind B}^{Mut}=ΔE_{Complex B}^{Mut}−ΔE_{Unfolded}^{Mut}−E_{ligand B}

ΔE_{Fold+Bind B}^{WT}=ΔE_{Complex B}^{WT}−ΔE_{Unfolded}^{WT}−E_{ligand B}

ΔE_{Fold+Bind A}^{Mut}=ΔE_{Complex A}^{Mut}−ΔE_{Unfolded}^{Mut}−E_{ligand A}

ΔE_{Fold+Bind A}^{WT}=ΔE_{Complex A}^{WT}−ΔE_{Unfolded}^{WT}−E_{ligand A}

The difference of the four above terms is equivalent to ΔΔΔE_{Bind B-Bind A}^{MUT-WT}:

ΔΔΔE_{Bind B-A}^{MUT-WT}=(ΔE_{Fold+Bind B}^{Mut}−ΔE_{Fold+Bind B}^{WT})−(ΔE_{Fold+Bind A}^{Mut}−ΔE_{Fold+Bind A}^{WT})

Mutants were then sorted on ΔΔE_{Bind B-Bind A}^{Mut-WT} and filtered with a fold cutoff of 15 kcal/mol in order to identify thiolase sequences with predicted differential selectivity as compared to wild type toward accommodating the butyryl group as opposed to the acetyl group. Sequences were then sorted on ΔΔE_{Bind B}^{Mut-WT} to allow identification of mutants for testing that were also predicted to accommodate the butyryl group more favorably than wild type, and not just resulting in improved differential specificity by accommodating both acetyl and butyryl groups more poorly than wild type, but with the acetyl worse than butyryl.

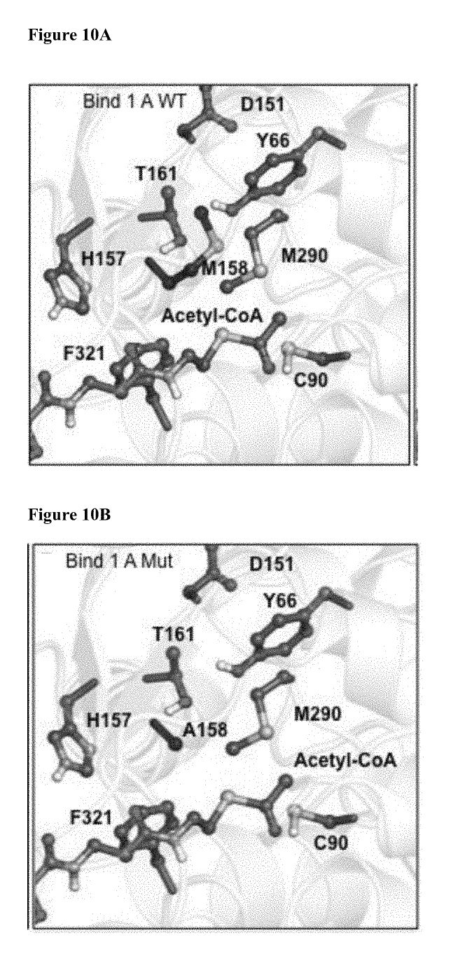

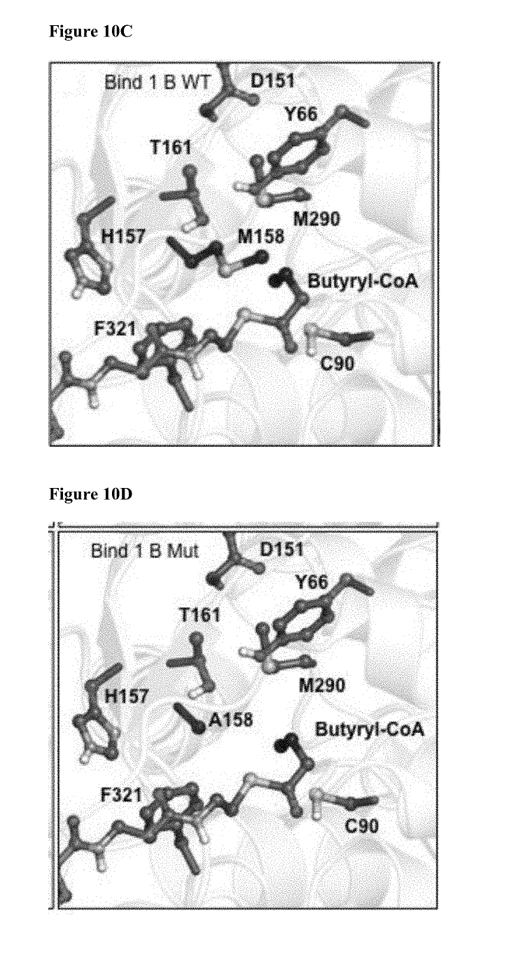

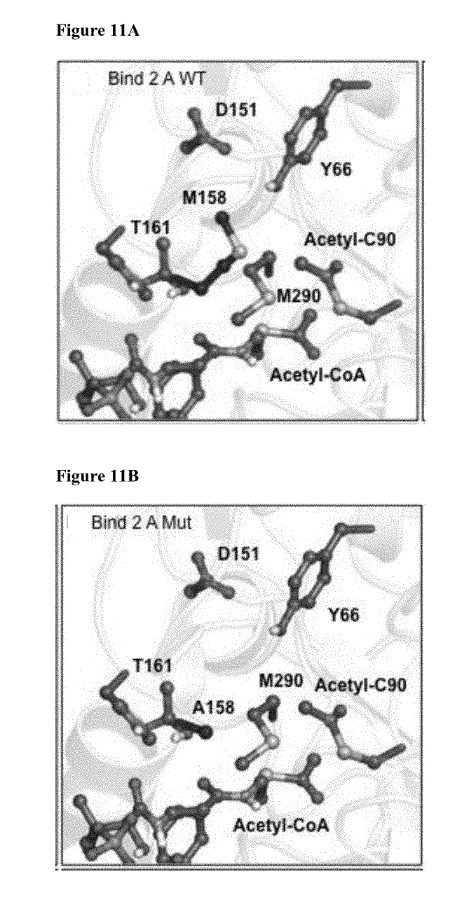

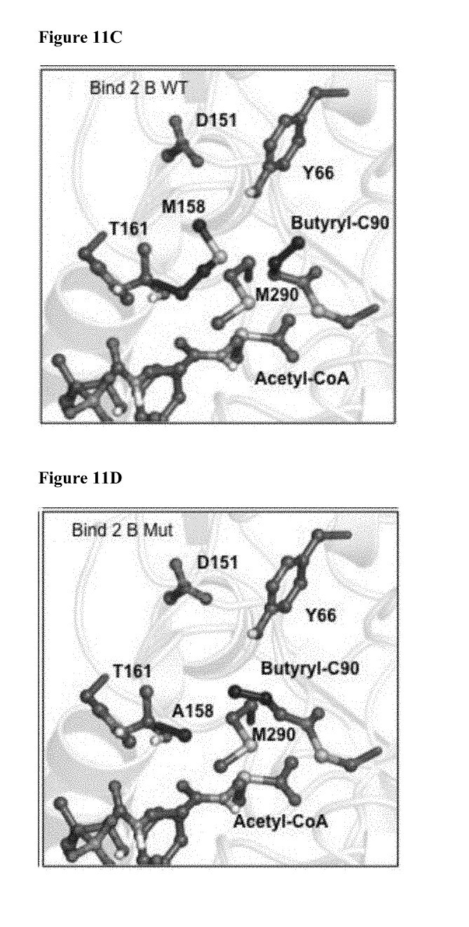

After optimized structures of mutants were computed and sequences were sorted, the dominant energetic interactions contributing to ΔΔΔE_{Bind B-Bind A}^{Mut-WT} for the top mutants were computed and analyzed. The top sets of four structures for each mutant for each binding event were manually inspected using this information. An example of the energetic breakdowns and four GMEC structures for one experimentally tested mutant in Bind 1 and Bind 2 is presented in FIGS. 10A-10D and 11A-11D and Tables VII and

TABLE VII
Dominant pairwise energetic interactions comprising ΔΔΔE_{Bind B-A Total}^{Mut-WT} for Bind
1 and corresponding to the four minimum energy structures shown in FIGS. 10A-10D

| Interaction | ΔΔE_{Bind B vdW}^{Mut-WT} | ΔΔE_{Bind A vdW}^{Mut-WT} | ΔΔΔE_{Bind B-A vdW}^{Mut-WT} | ΔΔΔE_{Bind B-A Total}^{Mut-WT} |
|---|---|---|---|---|
| (e) | (a) | (b) | (c) | (d) |
| A-158-Side | −2.65 | 0.73 | −3.37 | −3.32 |
| chain-C- | | | | |
| 1-Mercapto | | | | |
| Non-mobile- | −0.19 | 0.00 | −0.19 | −0.19 |
| C-Acyl | | | | |
| A-158-Side | 0.45 | 0.17 | 0.29 | 0.20 |
| chain-C- | | | | |
| 1- | | | | |
| PantoADP | | | | |
| A-158-Side | 0.49 | 0.21 | 0.29 | 0.26 |
| chain-C- | | | | |
| 1-CoACO | | | | |
| Total | −1.73 | 1.49 | −3.23 | −3.44 |

(a) ΔΔE_{Bind B vdW}^{Mut-WT} is the van der Waals (vdW) energy comprising the ΔΔE_{Bind B}^{Mut-WT term in Table VII.}
(b) ΔΔE_{Bind A vdW}^{Mut-WT} is the van der Waals energy comprising the ΔΔE_{Bind A}^{Mut-WT} term in Table VII.
(c) ΔΔΔE_{Bind B-A vdW}^{Mut-WT} is the van der Waals energy comprising the ΔΔΔE_{Bind B-A Total}^{Mut-WT} term in Table VII.
(d) ΔΔΔE_{Bind B-A Total}^{Mut-WT} is the total energy of each pairwise interaction comprising the sum of vdW, geometric and electrostatic interactions (latter two not displayed due to negligible contribution). The total in this column represents the ΔΔΔE_{Bind B-A}^{Mut-WT} term for M158A in the Bind 1 column of Table III. The four pairwise interactions listed are the only four interactions with |ΔΔΔE_{Bind B-A vdW}^{Mut-WT}| >0.1 kcal/mol.
(e) In Table VII, A-158-Side chain refers to the non-backbone atoms in residue 158, C-1-Mercapto refers to the atoms in the β-mercaptoethylamine group of the acetyl-CoA, C-1-PantoADP refers to the atoms in the pantothenic acid moiety of the acyl-CoA, C-1-CoACO refers to the two atoms in the acyl group carbonyl moiety of the acyl-CoA. Non-mobile refers to the atoms not included in the mobile region in the calculation (everything not shown as a ball and stick model in FIGS. 10A-10D).

TABLE VIII
Dominant Energetic Interactions Comprising ΔΔΔE_{Bind B-A}^{Mut-WT} in M158A Bind 2
and corresponding to the four minimum energy structures shown in FIGS. 11A-11D

| Interaction | ΔΔE_{Bind B vdW}^{Mut-WT} | ΔΔE_{Bind A vdW}^{Mut-WT} | ΔΔΔE_{Bind B-A vdW}^{Mut-WT} | ΔΔΔE_{Bind B-A Total}^{Mut-WT} |
|---|---|---|---|---|
| (e) | (a) | (b) | (c) | (d) |
| A-90-Acyl- | −1.33 | 0.00 | −1.34 | −1.30 |
| C-1- | | | | |
| Mercapto | | | | |
| A-90-CO- | −1.05 | −0.02 | −1.03 | −1.00 |
| C-1-Acyl | | | | |
| A-90-CO- | −0.21 | 0.00 | −0.21 | −0.27 |
| C-1-CoACO | | | | |
| Non-mobile- | −0.27 | 0.00 | −0.27 | −0.26 |
| C-Acyl | | | | |
| A-90-Acyl- | −0.18 | 0.00 | −0.19 | −0.21 |
| C-1-CoACO | | | | |
| A-90-Cys- | −0.19 | 0.00 | −0.18 | −0.16 |
| C-1-CoACO | | | | |
| A-90-Cys- | 0.20 | 0.00 | 0.20 | 0.20 |
| C-Acyl | | | | |
| Total | −1.69 | 1.38 | −3.07 | −3.03 |

(a) ΔΔE_{Bind B vdW}^{Mut-WT} is the van der Waals (vdW) energy comprising the ΔΔE_{Bind B}^{Mut-WT term in Table VIII.}
(b) ΔΔE_{Bind A vdW}^{Mut-WT} is the van der Waals energy comprising the ΔΔE_{Bind A}^{Mut-WT} term in Table VIII.
(c) ΔΔΔE_{Bind B-A vdW}^{Mut-WT} is the van der Waals energy comprising the ΔΔΔE_{Bind B-A Total}^{Mut-WT} term in Table VIII.
(d) ΔΔΔE_{Bind B-A Total}^{Mut-WT} is the total energy of each pairwise interaction comprising the sum of vdW, geometric and electrostatic interactions (latter two not displayed due to negligible contribution). The total in this column represents the ΔΔΔE_{Bind B-A}^{Mut-WT} term for M158A in the Bind 2 column of Table III. The seven pairwise interactions listed are the only seven interactions with |ΔΔΔE_{Bind B-A vdW}^{Mut-WT}| >0.1 kcal/mol.
(e) In Table VIII, A-90-Acyl refers to the aliphatic (non-carbonyl) portion of the acyl group on C90, A-90-CO, refers to the two atoms in the carbonyl portion of the acyl group on C90, A-90-Cys refers to the non-acyl, non-backbone portion of C90, C-1-Mercapto refers to the atoms in the β-mercaptoethylamine group of the acetyl-CoA, C-1-CoACO refers to the two atoms in the acetyl group carbonyl moiety of the acetyl-CoA, C-1-acyl refers to the non-carbonyl atoms in the acetyl group of acetyl-CoA. Non-mobile refers to the atoms not included in the mobile region in the calculation (everything not shown as a ball and stick model in FIGS. 11A-11D).

### Rotamer Library

For each mutant calculation, a dead-end elimination/A* based rotameric search was performed following the methodology of Lippow et al. (2007), with all residues within 4.75 Å of the mutated residue allowed to relax. For the rotameric search, a modified version of the Dunbrack rotamer library (Dunbrack and Cohen, 1997) was used in which χ₁ and χ₂ were expanded by ±10° from the crystal structure rotamers. The substrates and acylated cysteines were rotamerized in a manner to allow the acyl groups to rotate while keeping the rest of the atoms' positions fixed to that of the crystal structure. Using the atomic nomenclature introduced in FIG. 1D, for acetyl-CoA, acetyl-C, butyryl-CoA and butyryl-C the dihedrals Sγ-Cδ-Cε-Oδ and Cβ-Sγ-Cδ-Cε were allowed to rotate by ±10° from the corresponding crystal structure rotamers. For acetyl-CoA and acetyl-C, all atoms except Cδ, Oδ and Cε were held fixed. For butyryl-CoA and butyryl-C, the dihedrals Sγ-Cδ-Cε-Cζ and Cδ-Cε-Cζ-Cη were allowed to rotate in 30° increments, with atoms except Sγ, Cβ, Cδ, Oδ, Cε, and Cζ held fixed.

### Structural Analysis of M158A Mutation—Bind 1

FIGS. 10A-10D show the four global minimum energy structures that comprise the ΔΔΔE_{Bind B-A}^{Mut-WT} calculation for Bind 1 for the mutant chosen for in vitro characterization, M158A. Table VII shows the detailed pairwise energetic breakdowns of each of the terms comprising ΔΔΔE_{Bind B-A}^{Mut-WT}. According to Table VII, M158A is predicted to improve butyryl-CoA binding in the first binding event, hurt acetyl-CoA binding in the first step, improve accommodation of butyryl-Cys90 with acetyl-CoA bound in the second binding event, and disfavor accommodation of acetyl-Cys90 with acetyl-coA bound in the first binding event. The bulk of the improvement comes from the van der Waals (vdW) energy, particularly from the interaction of residue 158 with the mercapto group of acetyl/butyryl-CoA. Although the butyryl group between wild type and mutant takes on the same conformation, the M158 side chain takes on a conformation that clashes with the mercapto group. Residues shown with a ball-and-stick model are those included in the mobile region. FIGS. 10A-10D show that the majority of the mobile region does not locally rearrange in response to mutation or substrate binding. Only residues 290, 158, and 90 change conformation across the four figures. Nothing except the mutated residue changes conformation between FIGS. 10C and 10D, which represent the wild type and mutant bound to acetyl-CoA. The loss of favorable vdW contacts between the substrate and M158 as a result of paring M158 to a smaller alanine explains the positive value of 1.58 kcal/mol for ΔΔE_{Bind A}^{Mut-WT}, which represents the difference in binding energy between mutant and wild type bound to acetyl-coA. Note that to accommodate the bulkier butyryl group, the side chains of M290 and M158 take on conformations different from those in FIGS. 10C and 10D. In order to accommodate the butyryl group, the side chain of M158 takes on a less favorable conformation to butyryl binding. Mutating M158 to an alanine relieves this unfavorable interaction, explaining the favorable −1.86 kcal/mol value of ΔΔE_{Bind B}^{Mut-WT}.

### Structural Analysis of M158A Mutation—Bind 2

FIGS. 11A-11D show the four global minimum energy conformations that comprise the ΔΔΔE_{Bind B-A}^{Mut-WT} calculation for Bind 2 for the M158A structure. Compared to Bind 1, even fewer residues included in the mobile region change conformations between the four structures. In FIGS. 10C and 10D, as for Bind 1, the M158A mutation causes a loss of favorable vdW interactions between the M158 residue and the substrate, which explains the −1.49 kcal/mol value of ΔΔE_{Bind A}^{Mut-WT} for this binding event. FIGS. 10A and 10B represent the conformations of the mutant and wild type bound to acetyl-CoA with butyryl-C90. In FIG. 10A, the butyryl group of butyryl-C90 takes on a conformation that has an unfavorable interaction with the mercapto group of acetyl-CoA. Mutating M158 to alanine allows the butyryl group to relax to a more favorable conformation, which is worth −1.33 kcal/mol as shown in Table VIII.

### Discussion

The work described herein presents a rational design framework for increasing the thiolase selectivity ratio, which may correspond to, for example, the ratio of C6 to C4 condensation products. This framework was then applied to two related biosynthetic thiolases, PhbA from Z. ramigera and BktB from C. necator. In vivo, the synthesis of PHAs that are highly enriched for 3HHx (C6) was observed when the rationally selected mutants were employed. In vitro characterization of one of the most selective mutants (M158A) revealed a 10-fold reduction in activity for formation and breakdown of the C4 product with uncompromised thiolysis activity toward the C6 substrate as compared to the wild type enzyme.

Although the designed thiolase mutants described herein exhibited nearly a 10-fold improvement in the selectivity ratio, this increase was primarily driven by the reduced ability of the thiolase to synthesize AA (acetoacetyl-CoA, C4) and not improved ability to synthesize BA (3-oxo-hexanoyl-CoA, C6). The decreased synthesis of C4 products by the variants tested is consistent with in silico predictions, as all variants tested exhibited positive computed values of ΔΔE_{Bind B}^{Mut-WT} for both Bind 1 and Bind 2. From the in vitro kinetic characterization of the BktB M158A variant, the reduced rate of condensation of two acetyl-CoA substrates is consistent with reduced 3HB production within both pathway contexts (3HA and PHA).

The absence of significantly increased 3HH titers was not consistent with in silico predictions. With the exception of M288A/G, all variants tested had negative computed values of for either Bind 1 or Bind 2, meaning that each of the tested mutants were expected to preferentially accommodate the butyryl group in either the first (PhbA L377S/G and BktB M290S/A/G), second (PhbA L88A/G, PhbA M288S, BktB Y66N/V/T/A/G) or both (PhbA L88S, PhbA M157S/A/G, PhbA L377A, BktB M158S/A/G, and Y66Q/S) binding events.

It is possible that activities of downstream enzymes on the longer chain substrates limited 3HH production in vivo and activities of the downstream enzymes β-ketoacyl-CoA reductase and/or thioesterase and PHA polymerase) with the pathway acyl-CoA intermediates should be evaluated.

It was not possible to directly assay the rate of condensation between acetyl-CoA and butyryl-CoA in vitro to test whether mutants exhibited increased production of 3-oxo-hexanoyl-CoA (and subsequently 3-HH-CoA) in the absence of any potential confounding factors in vivo. It is possible to assay the thiolase in the thermodynamically favored direction (thiolysis) and observe higher activity of the M158A mutant with the C6 substrate. For example, when PHB synthesis was modeled in vitro, inclusion of the reductase enzyme was necessary to observe accumulation of the 3-ketoacyl-CoA condensation product (Burns et al., 2007). Both in vivo and in vitro the thiolase must be coupled to the reductase, and the substrate specificity and activity of the reductase enzyme will influence the behavior of the overall system. Indeed, a similar approach has also been used to model the kinetics of in vivo PHB accumulation (Leaf and Srienc, 1998; van Wegen et al, 2001). For this reason, in some embodiments, the system is examined whilst considering, at a minimum, the thiolase and reductase enzymes in combination.

Given that the butyryl group must be accommodated at two distinct locations within the active site, it is possible that multiple active-site mutations, rather than the point mutations tested in the work described herein, may improve C6 product titers beyond that of wild type. When the butyryl group is built onto the acetyl-CoA Cε carbon in structure 1M3Z (representing Bind 1) in its minimum energy planar zigzag conformation, the Cη atom of the butyryl group clashes directly with the backbone atoms of I379 and C378. The four non-polar, non-charged residues within 5 Å of the Cε carbon of acetyl-CoA in this structure, and the non-hydrogen atoms closest to the Cε carbon (side chain atom followed by distance in parenthesis) include M157 (S; 5.3 Å), M288 (S; 3.1 Å), A318 (CB; 4.5 Å), I379 (CB; 5.3 Å). Of these, M288 is the only residue that is directly in a position to clash with the butyryl group in a non-planar conformation, as the other three side chains are either too far away, or point away from the substrate. Similarly, if a butyryl group is built onto acetyl-C89 from structure 1DM3 (representing Bind 1) it clashes directly with G380. The three nonpolar, non-charged side chains within 5 Å of the acetyl carbon on acetylated C89 are L88 (Cβ; 3.2 Å), M157 (S; 4.9 Å away) and I379 (Cγ2; 4.5 Å). Of these, L88 and M157 are potentially positioned to clash with the C89 butyryl group in a non-planar conformation.

M157 is thus a PhbA active site residue that satisfied the energetic filtering criteria and was positioned to directly relieve a steric clash imposed by a bulky butyryl group in both binding events within the fixed backbone context of this work.

Applying the same analysis to BktB, if the butyryl group is added to the acetyl-CoA in its planar zigzag conformation (representing Bind 1), it clashes directly with the backbone atoms of C380 and I381. The nonpolar, non charged residues within 5 Å and their corresponding closest atoms are M158 (S; 4.6 Å), M290 (S; 3.1 Å), I381 (Cβ; 5.6 Å), A320 (Cβ; 4.2 Å), F321 (Cε2; 5.4 Å). Of these only M158 and M290 are positioned to directly clash with a non-planar butyryl C90. For BktB Bind 2, the butyryl group in its planar zigzag conformation clashes with the sidechains Y66 (the Cη of the butyryl group as lies 2.3 Å from the Cε2 of Y66), L89 (the C4 of the butyryl group as lies 2.8 Å from the Cδ2 of Le89), and G382. The nonpolar, non-charged residues within 5 Å are M158 (S; 5.0 Å), I352 (Cγ2; 4.6 Å), L89 (Cβ; 3.0 Å), Ile381 (Cγ2; 4.7 Å). Of these, L89, Y66, M158 and I352 are in orientations that could potentially clash with a butyryl C90. Only residues M290, M158, and Y66 met the energetic filtering criteria. Again, analogous to the PhbA case, M158 is the only side chain positioned to directly relieve a steric clash imposed by a butyryl group at both binding events.

The relative success of M157/M158 may be related to its location and orientation between the acyl group of the CoA substrate and the catalytic residue C89/C90. The fact that degradative thiolases, which are known to be able to accommodate >C6 substrates also have methionines at positions 290 and 158 suggests that mutations at other positions play a role in accommodating >C6 substrates (Fage et al., 2015; Modis and Wierenga, 1999b).

From a practical metabolic engineering standpoint, the thiolase variants identified in the work described herein, specifically the BktB M158A thiolase, may be useful in other pathways where the condensation of acetyl-CoA and different acyl-CoA species is required. In addition, it is shown herein that the thiolase can be used to modulate PHA polymer composition, resulting in PHAs that are highly enriched for medium-chain length monomers. Typically, PHA composition is modulated by process engineering such as novel feeding strategies and choice of feedstock, as wells as various strain engineering strategies to remove endogenous competing enzymes from native PHA synthesizing microbes. Using the thiolase to control PHA monomer composition opens up a new avenue for achieving the synthesis of PHAs with specific, desired properties for diverse applications.

### Example 2: Use of Thiolase Variants for the Production of 4-Methyl-Pentanol Pathway

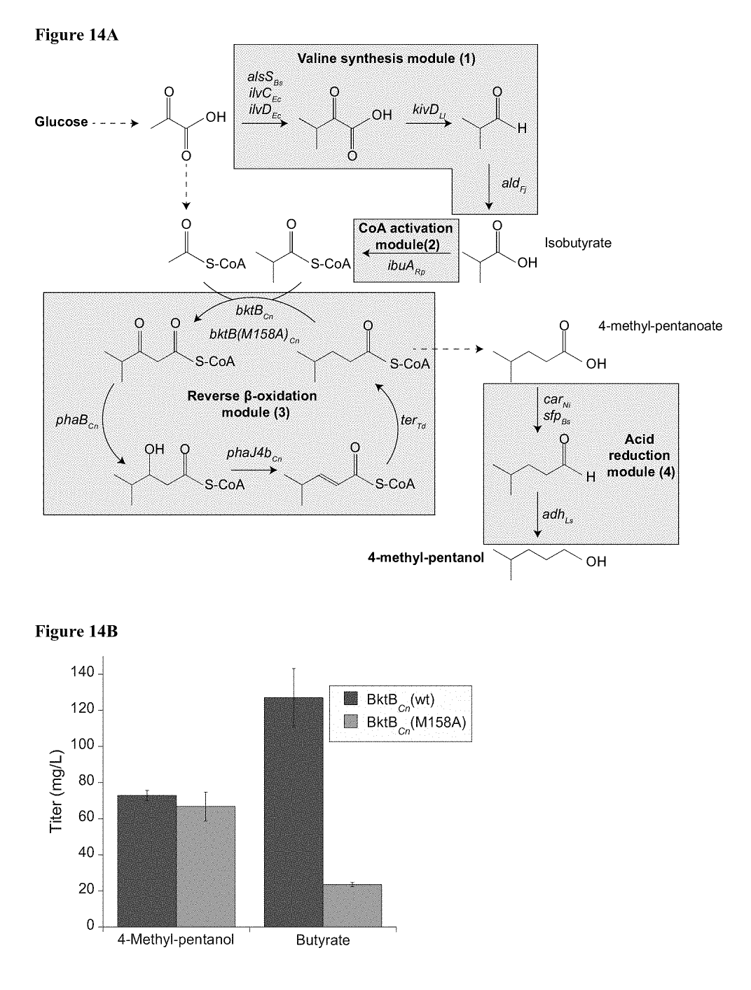

As discussed in Example 1, several example mutations were identified in BktB from C. necator that conferred increased specificity for C6 over C4 products (increased selectivity ratio). The BktB variants were then tested in the 4-methyl-penatanol (4MP) pathway, in which butyrate is an undesired byproduct. Butyrate production is the result of the condensation of two acetyl-CoA molecules, however in the production of 4MP, it is desired for the thiolase to condense isobutyryl-CoA with acetyl-CoA (FIG. 14A).

The M158A BktB variant was tested to determine if it had a reduced ability to form the acetoacetyl-CoA product, which would lower the amount of butyrate produced. Previously, Sheppard et. al 2014 showed that the wild type BktB from C. necator (BktB_{Cn}) was able to utilize isobutyryl-CoA as a substrate; however, it is unknown if the M158A BktB variant would show the same promiscuity. As such, the M158A BktB variant was tested within the 4MP pathway to determine if the enzyme could act upon isobutyryl-CoA and whether it would reduce the production of the butyrate byproduct.

Glucose was used as the starting substrate for 4MP production using the same plasmids, stain, and methods described in Sheppard et. al 2014. The 158A BktB variant was substituted for wild type BktB_{Cn} in one strain. The resulting 4MP titers indicated that the M158A BktB variant was able to use isobutyryl-CoA as a substrate and produce the 4MP product (FIG. 14B). The titers of 4MP achieved using the M158A BktB variant were similar to those achieved using the wild type BktB_{Cn}. Additionally, butryrate concentrations were measured and were found to be decreased 5-fold when the M158A BktB variant was used. These results validate that a thiolase with higher specificity can be utilized within this pathway to decrease the amount of byproducts formed by reducing an undesired side reaction (byproduct formation). However, the decrease in butyrate production did not result in an increase in 4MP titer, most likely due to a bottleneck elsewhere in the pathway.

### Methods

### 4-Methyl-Pentanol Production and Measurement

4-methyl-pentanol (4MP) production performed using identical methods, strains, and plasmids as were used in Sheppard et. al 2014. The M158A BktB variant was substituted for wild type BktB_{Cn} in the pET (BktB, Ter) (PhaB, PhaJ4b) plasmid. Briefly, MG1655 (DE3) ΔendA ΔrecA was transformed with plasmids containing all elements of the 4MP pathway and selected on appropriate antibiotics. Overnight cultures from individual colonies were grown at 30° C., and were subcultured 1:100 in 3 mL LB media containing 12 g/L glucose and appropriate antibiotics. Cultures were grown at 30° C. in 50 mL screw capped tubes and were induced for protein production with 0.5 mM IPTG at an OD600 nm between 0.5-0.9. 4MP production was carried out for 48 hours at 30° C.

After 48 hours of culturing, cell pellets were collected by centrifugation and compound production was measured from cell free supernatants. Compounds were extracted using a 1:1 volume of ethyl acetate by vortexing for 5 minutes followed by centrifugation for 5 minutes. The upper ethyl acetate layer was analyzed for 4MP and butyrate compounds using gas chromatography system. A WF-WAXms column was used for analysis and compounds were detected using a flame ionization detector (FID). Concentrations of 4MP and butyrate were determined by integrating FID chromatograms and comparison of area under the curve to standard curves of pure 4MP and butyrate at known concentrations, extracted and analyzed using identical media and procedure.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### REFERENCES

Bond-Watts B B, Bellerose R J, Chang M C Y. 2011. Enzyme mechanism as a kinetic control element for designing synthetic biofuel pathways. Nat. Chem. Biol. 7:222-7.
Brandl H, Gross R a, Lenz R W, Fuller R C. 1988. Pseudomonas oleovorans as a Source for novel Poly(beta-Hydroxyalkanoates). Appl. Environ. Microbiol. 54:1977-1982.
Brooks B R, Brooks C L, Mackerell A D, Nilsson L, Petrella R J, Roux B, Won Y, Archontis G, Bartels C, Boresch S, Caflisch A, Caves L, Cui Q, Dinner A R, Feig M, Fischer S, Gao J, Hodoscek M, Im W, Kuczera K, Lazaridis T, Ma J, Ovchinnikov V, Paci E, Pastor R W, Post C B, Pu J Z, Schaefer M, Tidor B, Venable R M, Woodcock H L, Wu X, Yang W, York D M, Karplus M. 2009. CHARMM: the biomolecular simulation program. J. Comput. Chem. 30:1545-614.
Budde C F, Riedel S L, Willis L B, Rha C, Sinskey A J. 2011. Production of poly(3-hydroxybutyrate-co-3-hydroxyhexanoate) from plant oil by engineered Ralstonia eutropha strains. Appl. Environ. Microbiol. 77:2847-2854.
Burns K L, Oldham C D, Thompson J R, Lubarsky M, May S W. 2007. Analysis of the in vitro biocatalytic production of poly-(β)-hydroxybutyric acid. Enzyme Microb. Technol. 41:591-599.
Cheong S, Clomburg J M, Gonzalez R. 2016a. Energy- and carbon-efficient synthesis of functionalized small molecules in bacteria using non-decarboxylative Claisen condensation reactions. Nat. Biotechnol. In press:1-8.
Cheong S, Clomburg J M, Gonzalez R. 2016b. Energy- and carbon-efficient synthesis of functionalized small molecules in bacteria using non-decarboxylative Claisen condensation reactions. Nat. Biotechnol. In press:1-8.
Dhamankar H, Tarasova Y, Martin C H, Prather K L J. 2014. Engineering E. coli for the biosynthesis of 3-hydroxy-γ-butyrolactone (3HBL) and 3,4-dihydroxybutyric acid (3,4-DHBA) as value-added chemicals from glucose as a sole carbon source. Metab. Eng. 25:72-81.
Dunn B J, Watts K R, Robbins T, Cane D E, Khosla C. 2014. Comparative analysis of the substrate specificity of trans-versus cis-acyltransferases of assembly line polyketide synthases. Biochemistry 53:3796-806.
Fage C D, Meinke J L, Keatinge-Clay A T. 2015. Coenzyme A-free activity, crystal structure, and rational engineering of a promiscuous B-ketoacyl thiolase from Ralstonia eutropha. J. Mol. Catal. B Enzym. 121:113-121.
Kim E J, Son H F, Kim S, Ahn J W, Kim K J. 2014a. Crystal structure and biochemical characterization of beta-keto thiolase B from polyhydroxyalkanoate-producing bacterium Ralstonia eutropha H16. Biochem. Biophys. Res. Commun. 444:365-369.
Kim E J, Son H F, Kim S, Ahn J W, Kim K J. 2014b. Crystal structure and biochemical characterization of beta-keto thiolase B from polyhydroxyalkanoate-producing bacterium Ralstonia eutropha H16. Biochem. Biophys. Res. Commun. 444:365-369.
Kim S, Clomburg J M, Gonzalez R. 2015. Synthesis of medium-chain length (C6-C10) fuels and chemicals via β-oxidation reversal in Escherichia coli. J. Ind. Microbiol. Biotechnol. 42:465-475.
Kursula P, Ojala J, Lambeir A-M, Wierenga R K. 2002. The Catalytic Cycle of Biosynthetic Thiolase: A Conformational Journey of an Acetyl Group through Four Binding Modes and Two Oxyanion Holes. Biochemistry 41:15543-15556.
Lippow S M, Wittrup K D, Tidor B. 2007. Computational design of antibody-affinity improvement beyond in vivo maturation. Nat. Biotechnol. 25:1171-6.
Martin C H, Dhamankar H, Tseng H-C, Sheppard M J, Reisch C R, Prather K L J. 2013a. A platform pathway for production of 3-hydroxyacids provides a biosynthetic route to 3-hydroxy-γ-butyrolactone. Nat. Commun. 4:1414.
Martin C H, Dhamankar H, Tseng H-C, Sheppard M J, Reisch C R, Prather K L J. 2013b. A platform pathway for production of 3-hydroxyacids provides a biosynthetic route to 3-hydroxy-γ-butyrolactone. Nat. Commun. 4:1414.
McMahon M D, Prather K L J. 2014. Functional screening and in vitro analysis reveal thioesterases with enhanced substrate specificity profiles that improve short-chain fatty acid production in Escherichia coli. Appl. Environ. Microbiol. 80:1042-1050.
Meriläinen G, Poikela V, Kursula P, Wierenga R K. 2009. The thiolase reaction mechanism: the importance of Asn316 and His348 for stabilizing the enolate intermediate of the Claisen condensation. Biochemistry 48:11011-25.
Meriläinen G, Schmitz W, Wierenga R K, Kursula P. 2008. The sulfur atoms of the substrate CoA and the catalytic cysteine are required for a productive mode of substrate binding in bacterial biosynthetic thiolase, a thioester-dependent enzyme. FEBS J. 275:6136-48.
Modis Y, Wierenga R K. 1999a. A biosynthetic thiolase in complex with a reaction intermediate: the crystal structure provides new insights into the catalytic mechanism. Structure 7:1279-90.
Modis Y, Wierenga R K. 2000. Crystallographic analysis of the reaction pathway of Zoogloea ramigera biosynthetic thiolase. J. Mol. Biol. 297:1171-82.
Modis Y, Wierenga R K. 1999b. A biosynthetic thiolase in complex with a reaction intermediate: the crystal structure provides new insights into the catalytic mechanism. Structure 7:1279-1290.
Sheppard M J, Kunjapur A M, Wenck S J, Prather K L J. 2014. Retro-biosynthetic screening of a modular pathway design achieves selective route for microbial synthesis of 4-methyl-pentanol. Nat. Commun. 5:5031.
Slater S, Houmiel K L, Tran M, Timothy A, Taylor N B, Padgette S R, Kenneth J, Mitsky T A, Gruys K J. 1998. Multiple β-Ketothiolases Mediate Poly (β-Hydroxyalkanoate) Copolymer Synthesis in Ralstonia eutropha. J. Bacteriol. 180:1979-1987.
Stubbe J, Tian J. 2003. Polyhydroxyalkanoate (PHA) hemeostasis: the role of PHA synthase. Nat. Prod. Rep. 20:445-457.
Tseng H-C, Martin C H, Nielsen D R, Prather K L J. 2009. Metabolic engineering of Escherichia coli for enhanced production of (R)- and (S)-3-hydroxybutyrate. Appl. Environ. Microbiol. 75:3137-45.
Yuan W, Jia Y, Tian J, Snell K D, Müh U, Sinskey A J, Lambalot R H, Walsh C T, Stubbe J. 2001. Class I and III polyhydroxyalkanoate synthases from Ralstonia eutropha and Allochromatium vinosum: characterization and substrate specificity studies. Arch. Biochem. Biophys. 394:87-98.

1. A thiolase variant comprising:
an amino acid substitution at a position corresponding to M158 and/or M290 of SEQ ID NO: 2,
wherein the thiolase variant has greater than 40% amino acid identity to SEQ ID NO: 1 or wherein the region within 10 angstroms of the active site of the thiolase variant has greater than 75% amino acid identity to the corresponding region of SEQ ID NO:1.

2. A thiolase variant comprising
(a) an amino acid sequence provided by SEQ ID NO: 1 and wherein the thiolase variant comprises a substitution at one or more positions corresponding to V57, R88, L89, S92, L94, A148, L149, H150, D151, M158, M290, N318, I352, T355, M379, I381, or M65 of SEQ ID NO: 2;
(b) an amino acid sequence provided by SEQ ID NO: 2 and wherein the thiolase variant comprises a substitution at one or more positions corresponding to V57, R88, L89, S92, L94, A148, L149, H150, D151, M158, M290, N318, A320, F321, I352, T355, M379, I381, I387, or Y66 of SEQ ID NO: 2;
(c) an amino acid sequence provided by SEQ ID NO: 3 and wherein the thiolase variant comprises a substitution at one or more positions corresponding to M156 or M287 of SEQ ID NO: 3; or
(d) an amino acid sequence provided by SEQ ID NO: 4 and wherein the thiolase variant comprises a substitution at one or more positions corresponding to M157 or M289 of SEQ ID NO: 4

3.-6. (canceled)

7. The thiolase variant of claim 2, wherein the amino acid substitution is selected from the group consisting of L89S, M158A, M158G, M158S, M290G, and M290S of SEQ ID NO: 2.

8. (canceled)

9. (canceled)

10. The thiolase variant of claim 1, wherein the thiolase variant has an enhanced selectivity ratio as compared to a thiolase that does not comprise the amino acid substitution; optionally wherein the enhanced selectivity ratio corresponds to production of an increased ratio of one or more C6 products relative to one or more C4 products.

11. (canceled)

12. The thiolase variant of claim 10, wherein the C6 product is 3HH-CoA and/or the C4 product is 3HB-CoA.

13. (canceled)

14. A nucleic acid encoding the thiolase variant of claim 1.

15. A vector comprising the nucleic acid of claim 14.

16. A cell that recombinantly expresses the thiolase variant of claim 1.

17. The cell of claim 16, wherein the cell further recombinantly expresses any one or more enzymes selected from the group consisting of:
(a) a Coenzyme A activator enzyme, optionally Pct from Megasphaera elsdenii;
(b) a NADPH dependent reductase, optionally PhaB from Cupriavidus necator; and
(c) a thioesterase, optionally TesB from Escherichia coli.

18.-20. (canceled)

21. The cell of claim 16, wherein the cell further recombinantly expresses any one or more enzymes selected from the group consisting of:
(a) an enoyl-CoA reductase, optionally Ter from Treponema denticola;
(b) an enoyl-CoA dehydratase, optionally PhaJ4b from Cupriavidus necator;
(c) a PHA polymerase, optionally PhaC2 from Rhodococcus aetherivorans;
(d) an alcohol or aldehyde dehydrogenase;
(e) a carboxylic acid reductase, optionally Car from Mycobacterium marinum; and
(f) a hydroxylase, optionally A1kBGT from Pseudomonas putida.

22.-26. (canceled)

27. The cell of claim 16, wherein the cell is a bacterial cell, a fungal cell, a plant cell, an insect cell, or an animal cell.

28. The cell of claim 27, wherein the cell is a bacterial cell, optionally an Escherichia coli cell.

29. (canceled)

30. The cell of claim 16, wherein the cell produces a 3-hydroxalkonic acid (3HA), carboxylic acid, dicarboxylic acid, methyl ketone, hydroxy-carboxylic acid, PHA, keto-acid, aldehyde, alcohol, or alkane.

31. The cell of claim 30, wherein the cell produces a desired 3HA and a byproduct HA, and wherein the ratio of the desired 3HA to byproduct HA is greater than 1.

32. The cell of claim 31, wherein the desired 3HA is 3-oxo-hexanoyl-CoA, 3-hydroxy-hexanoic acid, or 3-hydroxy-hexanoate (3HHx) and/or the byproduct 3HA is acetoacetyl-CoA or 3-hydroxbuytric acid (3HB); optionally, wherein the desired 3HA is 4-methyl pentanol and/or the byproduct 3HA is butyrate.

33.-35. (canceled)

36. A cell culture or supernatant collected from culturing one or more cells of claim 16; optionally wherein the cell culture or supernatant contains at least 0.1 g/L 3HHx or 0.1 g/L 4-methyl pentanol.

37. (canceled)

38. The cell culture or supernatant of claim 36, wherein the 3HHx or the 4-methyl pentanol is further purified from the cell culture or supernatant.

39. (canceled)

40. (canceled)

41. A method comprising culturing the cell of claim 16 in cell culture medium.

42. The method of claim 41, wherein glucose, butyrate, and/or isobutyrate is added to the cell culture medium.

43. (canceled)

44. (canceled)

45. A method for producing 3-hydroxy-hexanoic acid, 3-hydroxy-hexanoate, or 4-methyl pentanol comprising culturing the cell of claim 16.

46. (canceled)


SEQUENCE LISTING (61 sequences)

SEQ ID NO: 1 - protein, 394 aa, Z. ramigera
MSTPSIVIASAARTAVGSFNGAFANTPAHELGATVISAVLERAGVAAGEVNEVILGQVLPAGEGQNPARQAAMKAGVPQEATAWGMNQLCGSGLRAVALGMQQIATGDASIIVAGGMESMSMAPHCAHLRAGGVKMGDFKMIDTMIKDGLTDAFYGYHMGTTAENVAKQWQLSRDEQDAFAVASQNKAEAAQKDGRFKDEIVPFIVKGRKGDITVDADEYIRHGATLDSMAKLRPAFDKEGTVTAGNASGLNDGAAAALLMSEAEASRRGIQPLGRIVSWATVGVDPKVMGTGPIPASRKALERAGWKIGDLDLVEANEAFAAQACAVNKDLGWDPSIVNVNGGAIAIGHPIGASGARILNTLLFEMKRRGARKGLATLCIGGGMGVAMCIESL

SEQ ID NO: 2 - protein, 394 aa, C. necator
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 3 - protein, 390 aa, Rhodospirillum rubrum
MTDIVIAGATRTPVGTFNGGLSGLPAHALGEIVIREVLRRAKVEAGEVDEVVLGQILTAGQGQNPARQAAVNAGIPVEKTAYGINQLCGSGLRTVALGFQAITLGDADIVVAGGQESMSMAPHATYLRSGIKMGTTELVDTMLKDGLWDAFHGYHMGTTAENIAGKWQISREDQDLFSAGSQNKAEAAIAAGRFKDEIVPVTVKGRKGDIVIDTDEHPKAGVTPESLAKLRPAFSKDGTVTAGNASGINDGAAALVLMSEANAAKRGVAPLARIVSWATAGVDPAIMGTGPIPATRKALEKAGWSIDDLDLIEANEAFAAQALAVNKDLGWDPAKINVNGGAIALGHPVGASGARILVTLLHEMIKRDAKKGLATLCIGGGMGIALCVER

SEQ ID NO: 4 - protein, 393 aa, C. necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 5 - DNA, 28 bp, Artificial Sequence
  Synthetic Polynucleotide
ggcggtcacgcccgcgtggatgcgatgg

SEQ ID NO: 6 - DNA, 28 bp, Artificial Sequence
  Synthetic Polynucleotide
ccatcgcatccacgcgggcgtgaccgcc

SEQ ID NO: 7 - DNA, 28 bp, Artificial Sequence
  Synthetic Polynucleotide
ccatcgcatccacgggggcgtgaccgcc

SEQ ID NO: 8 - DNA, 28 bp, Artificial Sequence
  Synthetic Polynucleotide
ggcggtcacgcccccgtggatgcgatgg

SEQ ID NO: 9 - DNA, 31 bp, Artificial Sequence
  Synthetic Polynucleotide
cccttccatcgcatccacagcggcgtgaccg

SEQ ID NO: 10 - DNA, 31 bp, Artificial Sequence
  Synthetic Polynucleotide
cggtcacgccgctgtggatgcgatggaaggg

SEQ ID NO: 11 - DNA, 28 bp, Artificial Sequence
  Synthetic Polynucleotide
ggacccgaaggcggcgggcatcggcccg

SEQ ID NO: 12 - DNA, 28 bp, Artificial Sequence
  Synthetic Polynucleotide
cgggccgatgcccgccgccttcgggtcc

SEQ ID NO: 13 - DNA, 28 bp, Artificial Sequence
  Synthetic Polynucleotide
ggacccgaaggcggggggcatcggcccg

SEQ ID NO: 14 - DNA, 28 bp, Artificial Sequence
  Synthetic Polynucleotide
cgggccgatgccccccgccttcgggtcc

SEQ ID NO: 15 - DNA, 28 bp, Artificial Sequence
  Synthetic Polynucleotide
tggacccgaaggcgagcggcatcggccc

SEQ ID NO: 16 - DNA, 28 bp, Artificial Sequence
  Synthetic Polynucleotide
gggccgatgccgctcgccttcgggtcca

SEQ ID NO: 17 - DNA, 28 bp, Artificial Sequence
  Synthetic Polynucleotide
gccgcgcgacatggctctgggccgcgtc

SEQ ID NO: 18 - DNA, 28 bp, Artificial Sequence
  Synthetic Polynucleotide
gacgcggcccagagccatgtcgcgcggc

SEQ ID NO: 19 - DNA, 28 bp, Artificial Sequence
  Synthetic Polynucleotide
gccgcgcgacatgggtctgggccgcgtc

SEQ ID NO: 20 - DNA, 28 bp, Artificial Sequence
  Synthetic Polynucleotide
gacgcggcccagacccatgtcgcgcggc

SEQ ID NO: 21 - DNA, 25 bp, Artificial Sequence
  Synthetic Polynucleotide
ccgcgcgacatgaatctgggccgcg

SEQ ID NO: 22 - DNA, 25 bp, Artificial Sequence
  Synthetic Polynucleotide
cgcggcccagattcatgtcgcgcgg

SEQ ID NO: 23 - DNA, 29 bp, Artificial Sequence
  Synthetic Polynucleotide
gccgcgcgacatgcagctgggccgcgtcg

SEQ ID NO: 24 - DNA, 29 bp, Artificial Sequence
  Synthetic Polynucleotide
cgacgcggcccagctgcatgtcgcgcggc

SEQ ID NO: 25 - DNA, 28 bp, Artificial Sequence
  Synthetic Polynucleotide
gccgcgcgacatgagtctgggccgcgtc

SEQ ID NO: 26 - DNA, 28 bp, Artificial Sequence
  Synthetic Polynucleotide
gacgcggcccagactcatgtcgcgcggc

SEQ ID NO: 27 - DNA, 28 bp, Artificial Sequence
  Synthetic Polynucleotide
gccgcgcgacatgactctgggccgcgtc

SEQ ID NO: 28 - DNA, 28 bp, Artificial Sequence
  Synthetic Polynucleotide
gacgcggcccagagtcatgtcgcgcggc

SEQ ID NO: 29 - DNA, 28 bp, Artificial Sequence
  Synthetic Polynucleotide
gccgcgcgacatggttctgggccgcgtc

SEQ ID NO: 30 - DNA, 28 bp, Artificial Sequence
  Synthetic Polynucleotide
gacgcggcccagaaccatgtcgcgcggc

SEQ ID NO: 31 - DNA, 17 bp, Artificial Sequence
  Synthetic Polynucleotide
gcagcctgaaggatgtg

SEQ ID NO: 32 - DNA, 17 bp, Artificial Sequence
  Synthetic Polynucleotide
gcgccttcaccgtgatc

SEQ ID NO: 33 - DNA, 29 bp, Artificial Sequence
  Synthetic Polynucleotide
gctagcgaattcctacaaggagaacaaac

SEQ ID NO: 34 - DNA, 22 bp, Artificial Sequence
  Synthetic Polynucleotide
ctagaggatccccgggctcgag

SEQ ID NO: 35 - DNA, 33 bp, Artificial Sequence
  Synthetic Polynucleotide
aacctgtattttcagggcatgactcagcgcatt

SEQ ID NO: 36 - DNA, 33 bp, Artificial Sequence
  Synthetic Polynucleotide
gctcgagaattccatggctcagcccatatgcag

SEQ ID NO: 37 - DNA, 28 bp, Artificial Sequence
  Synthetic Polynucleotide
gggtctcgccacggcctgcatcggcggc

SEQ ID NO: 38 - DNA, 28 bp, Artificial Sequence
  Synthetic Polynucleotide
gccgccgatgcaggccgtggcgagaccc

SEQ ID NO: 39 - DNA, 28 bp, Artificial Sequence
  Synthetic Polynucleotide
gggtctcgccacgggctgcatcggcggc

SEQ ID NO: 40 - DNA, 28 bp, Artificial Sequence
  Synthetic Polynucleotide
gccgccgatgcagcccgtggcgagaccc

SEQ ID NO: 41 - DNA, 28 bp, Artificial Sequence
  Synthetic Polynucleotide
gggtctcgccacgagctgcatcggcggc

SEQ ID NO: 42 - DNA, 28 bp, Artificial Sequence
  Synthetic Polynucleotide
gccgccgatgcagctcgtggcgagaccc

SEQ ID NO: 43 - DNA, 28 bp, Artificial Sequence
  Synthetic Polynucleotide
gcccgagccgcaagcctggttcatgccc

SEQ ID NO: 44 - DNA, 28 bp, Artificial Sequence
  Synthetic Polynucleotide
gggcatgaaccaggcttgcggctcgggc

SEQ ID NO: 45 - DNA, 28 bp, Artificial Sequence
  Synthetic Polynucleotide
gcccgagccgcaaccctggttcatgccc

SEQ ID NO: 46 - DNA, 28 bp, Artificial Sequence
  Synthetic Polynucleotide
gggcatgaaccagggttgcggctcgggc

SEQ ID NO: 47 - DNA, 30 bp, Artificial Sequence
  Synthetic Polynucleotide
ggcccgagccgcaactctggttcatgcccc

SEQ ID NO: 48 - DNA, 30 bp, Artificial Sequence
  Synthetic Polynucleotide
ggggcatgaaccagagttgcggctcgggcc

SEQ ID NO: 49 - DNA, 28 bp, Artificial Sequence
  Synthetic Polynucleotide
ctacggctaccacgcgggcacgaccgcc

SEQ ID NO: 50 - DNA, 28 bp, Artificial Sequence
  Synthetic Polynucleotide
ggcggtcgtgcccgcgtggtagccgtag

SEQ ID NO: 51 - DNA, 28 bp, Artificial Sequence
  Synthetic Polynucleotide
ctacggctaccacgggggcacgaccgcc

SEQ ID NO: 52 - DNA, 28 bp, Artificial Sequence
  Synthetic Polynucleotide
ggcggtcgtgcccccgtggtagccgtag

SEQ ID NO: 53 - DNA, 31 bp, Artificial Sequence
  Synthetic Polynucleotide
gccttctacggctaccacagcggcacgaccg

SEQ ID NO: 54 - DNA, 31 bp, Artificial Sequence
  Synthetic Polynucleotide
cggtcgtgccgctgtggtagccgtagaaggc

SEQ ID NO: 55 - DNA, 28 bp, Artificial Sequence
  Synthetic Polynucleotide
cgatcccaaggtcgcgggcaccggcccg

SEQ ID NO: 56 - DNA, 28 bp, Artificial Sequence
  Synthetic Polynucleotide
cgggccggtgcccgcgaccttgggatcg

SEQ ID NO: 57 - DNA, 28 bp, Artificial Sequence
  Synthetic Polynucleotide
cgatcccaaggtcgggggcaccggcccg

SEQ ID NO: 58 - DNA, 28 bp, Artificial Sequence
  Synthetic Polynucleotide
cgggccggtgcccccgaccttgggatcg

SEQ ID NO: 59 - DNA, 28 bp, Artificial Sequence
  Synthetic Polynucleotide
cgtcgatcccaaggtcagcggcaccggc

SEQ ID NO: 60 - DNA, 28 bp, Artificial Sequence
  Synthetic Polynucleotide
gccggtgccgctgaccttgggatcgacg

SEQ ID NO: 61 - DNA, 1704 bp, Artificial Sequence
  Synthetic Polynucleotide
gatatacatatggcacaggcacgtaccgttattggtgaaagcgttgaagaaagcattggtggtggtgaagatgttgcaccgcctcgtctgggtccggcagttggtgcactggcagatgtttttggtcatggtcgtgcagttgcacgtcatggtgttagctttggtcgtgaactggcaaaaattgcagttggtcgtagcaccgttgcaccggcaaaaggtgatcgtcgttttgcagatagcgcatggtcagcaaatccggcatatcgtcgcctgggtcagacctatctggcagcaaccgaagcagttgatggtgttgttgatgaagtgggtcgtgcaattggtccgcgtcgtaccgcagaagcacgttttgccgcagatattctgaccgcagcactggcaccgaccaattatctgtggaccaatccggcagcactgaaagaagcatttgataccgcaggtctgagcctggcacgtggcaccaaacattttgttagcgatctgattgaaaatcgtggtatgccgagcatggttcagcgtggtgcatttaccgttggtaaagatctggcagttacaccgggtgcagttattagccgtgatgaagttgccgaagttctgcagtataccccgaccaccgaaaccgttcgtcgtcgtccggttctggttgttccgcctccgattggtcgttattactttctggatctgcgtccgggtcgtagctttgttgaatatagtgttggtcgtggcctgcagacctttctgctgagctggcgtaatccgaccgcagaacagggtgattgggattttgatacctatgcaggtcgtgttattcgtgcaatcgatgaagttcgtgaaatcaccggtagtgatgatgttaatctgattggtttttgtgccggtggtattattgcaaccaccgttctgaatcacctggcagcccagggtgatacccgtgttcatagcatggcctatgcagttaccatgctggattttggtgatccggcactgctgggtgcatttgcccgtcctggtctgattcgttttgccaaaggtcgtagccgtcgtaaaggtattattagcgcacgtgatatgggtagcgcatttacctggatgcgtccgaatgatctggtttttaactatgtggtgaacaactatctgatgggtcgtacccctcctgcctttgatattctggcatggaatgatgatggtacaaatctgcctggtgccctgcatggccagtttctggatatttttcgtgataatgttctggtggaaccgggtcgtctggccgttctgggtacaccggttgatctgaaaagcattaccgttccgacctttgtgagcggtgcaattgccgatcatctgaccgcgtggcgtaattgttatcgtaccacacagctgctgggaggtgaaaccgaatttgcactgagctttagcggtcatattgcaagcctggttaatcctccgggtaatccgaaagcacattattggaccggtggcacaccgggtccggatcctgatgcatggctggaaaatgcagaacgtcagcagggtagttggtggcaggcctggtcagattgggttctggcacgcggtggcgaagaaacagcagcaccggatgcaccgggtagtgcacagcatcctgcactggatgccgcaccgggtcgctatgttcgtgatctgcctgcaggttaactcgagtctggt